(19) United States
(12) Patent Application Publication
(10) Pub. No.: US 2009/0286839 A1
(43) Pub. Date: Nov. 19, 2009

(54) PHYTONUTRIENT COMPOSITIONS FROM MUSHROOMS OR FILAMENTOUS FUNGI AND METHODS OF USE

(75) Inventors: ROBERT B. BEELMAN, UNIVERSITY PARK, PA (US); N. JOY DUBOST, STATE COLLEGE, PA (US); DEVIN G. PETERSON, UNIVERSITY PARK, PA (US); MARVIN HAUSMAN, STEVENSON, WA (US)

Correspondence Address: MCKEE, VOORHEES & SEASE, P.L.C.;ATTN: PENNSYLVANIA STATE UNIVERSITY, 801 GRAND AVENUE, SUITE 3200, DES MOINES, IA, 50309-2721, US

(73) Assignee: THE PENN STATE RESEARCH FOUNDATION, University Park, PA (US)

(21) Appl. No.: 12/502,677

(22) Filed: Jul. 14, 2009

Related U.S. Application Data
(62) Division of application No. 11/686,033, filed on Mar. 14, 2007, now abandoned.
(60) Provisional application No. 60/782,204, filed on Mar. 14, 2006.

(51) Int. Cl. A61K 31/4164; C07D 233/02

(52) U.S. Cl. 514/385; 548/300.1

(57) ABSTRACT

This invention is directed to treating disease states or conditions associated with the treatment and prevention of neurodegeneration and neurodegenerative disease states, and treatment of radiation damage. The invention relates to novel phytonutrient compositions and compounds comprising L-ergothioneine and/or selenium. The invention also provides a method of administering these compositions and combinations to humans or animals in need thereof.

### CROSS-REFERENCE TO RELATED APPLICATIONS

This is a divisional application of U.S. patent application Ser. No. 11/686,033 filed Mar. 14, 2007, which application claims priority under 35 U.S.C. § 119 of Provisional Application Ser. No. 60/782,204 filed Mar. 14, 2006, and which applications are hereby incorporated by reference in their entirety.

### GRANT REFERENCE

Work for this invention was funded in part by a grant from the United States Department of Agriculture Grant Numbers are Hatch Act Project No. PEN03774 and Hatch Act Project No. PEN04092. The Government may have certain rights in this invention.

### FIELD OF THE INVENTION

The present invention relates generally to the fields of pharmacology and neurology and the application of phytonutrient compositions and combinations for the treatment and prevention of neurodegeneration and treatment of radiation damage in humans and animals. More specifically, this invention provides methods for the use of ergothioneine and/or selenium for neuroprotection.

### BACKGROUND OF THE INVENTION

Research in the area of phytonutrients in various food materials has shown that specific plant components may have a positive effect on health. Recent publications on plant natural antioxidant products, for example, have been associated with human biological function. Antioxidants present in the diet can act as possible protective agents against oxidative stress and damage.

Injuries or trauma of various kinds to the central nervous system (CNS) or the peripheral nervous system (PNS) can produce profound and long-lasting neurological and/or psychiatric symptoms and disorders. One form that this takes is the progressive death of neurons or other cells of the CNS, i.e., neurodegeneration or neuronal degeneration. Neuronal degeneration as a result of, for example; Alzheimer's disease, multiple sclerosis, cerebral-vascular accidents (CVAs) stroke, traumatic brain injury, spinal cord injuries, degeneration of the optic nerve, e.g., ischemic optic neuropathy or retinal degeneration and other CNS disorders present both medical and public health concerns by virtue of both its high incidence and the implications of long-term aftereffects and care. Animal studies and clinical trials have shown that amino acid transmitters, oxidative stress and inflammatory reactions contribute strongly to cell death in these conditions.

In many chronic neurodegenerative conditions, inflammation and oxidative stress are key components of the pathology. These conditions include Alzheimer's disease (AD). This disease is characterized by the accumulation of neurofibrillary tangles and senile plaques, and a widespread, progressive degeneration of neurons in the brain. Senile plaques are rich in amyloid precursor protein (APP) that is encoded by the APP gene located on chromosome 21.

Parkinson's disease (PD) is a progressive neurodegenerative disorder characterized by a dysfunction of movement consisting of akinesia, rigidity, tremor and postural abnormalities. This disease has been associated with the loss of nigro-striatal dopaminergic neuronal integrity and functionality as evidenced by substantial loss of dopaminergic neurons in substantia nigra pars compacta (SNpc) (See, Pakkenberg et al. (1991) J. Neurol. Neurosurg. Psychiat. 54:30-33), and a decrease in content, synaptic and vesicular transporters of dopamine in the striatum (see, for example, Guttnan et al. (1997) Neurology 48:1578-1583).

Death of neurons and supporting cells in the CNS or peripheral nervous system (PNS) of mammals including humans as a result of trauma, injury of many kinds, ischemia, metabolic derangements, e.g., diabetes hypoxia, toxins or surgical intervention causes both acute and chronic and progressive loss of function and disability. Thus there is a need for the development of methods and compounds that can protect the cells of the mammalian nervous system from this degeneration, i.e., are neuroprotective.

Mushrooms have long been known for their nutritious benefits. They are an excellent source of selenium, riboflavin, pantothenic acid, copper, niacin, potassium and phosphorous. In particular, selenium is needed for the proper function of important antioxidants which works to reduce the levels of damaging free radicals in the body. Selenium is a necessary cofactor of one of the body's most important internally produced antioxidants, glutathione peroxidase, and also works synergistically with vitamin E in numerous vital antioxidant systems throughout the body. These powerful antioxidant actions make selenium helpful not only against cancer by protecting cells from cancer-causing toxins, but in decreasing asthma and arthritis symptoms and in the prevention of heart disease. In addition, selenium is involved in DNA repair, yet another way in which adequate intake of this mineral is associated with a reduced risk for cancer.

L-ergothioneine is a phytonutrient and has been identified in mushrooms. It is a naturally occurring antioxidant that is very stable in the body. It is synthesized in fungi and microorganisms, and present in both plants and animals. Mammals and humans are unable to synthesize L-ergothioneine and must obtain it from dietary sources. It is readily absorbed and is active in most mammalian tissues, concentrating especially in the liver, where it prevents certain types of free-radical-induced damage to cell membranes and organelles. For example, exogenous L-ergothioneine has been shown to prevent lipid peroxidation by toxic compounds in the liver tissue of rats. In a recent study comparing the inhibition of lip peroxide (“LPO”) formation by various compounds in mouse liver, L-ergothioneine both inhibited LPO formation and enhanced the decomposition of existing LPO.

Additionally, L-ergothioneine serves as an antioxidant and a cellular protector against oxidative damage. The antioxidant properties of L-ergothioneine include: a scavenger of strong oxidants; chelation of various divalent metallic cations; and plays a key role in the oxidation of various hemoproteins. L-ergothioneine has been shown to inhibit the damaging effects caused by the oxidation of iron-containing compounds, such as hemoglobin and myoglobin. These molecules are important in the body as carriers of oxygen, but because they contain divalent iron, they can interact with hydrogen peroxide via the Fenton reaction to produce the even more damaging hydroxyl radical. This has been suggested as a mechanism by which damage occurs during so-called reperfusion injury.

Although L-ergothioneine does not directly scavenge superoxide anion or hydrogen peroxide, it contributes to the control of these free radicals by participating in the function of superoxide dismutase and glutathione peroxidase. Its protective effects on cell membranes and other organelles are of benefit in acute and chronic toxicity as well as in infectious diseases, because common pathogenic biomechanisms are active in both of these processes. Ergothioneine in any form would be useful in the invention, including natural, semisynthetic, bioengineered, synthetic, extracted and combinations thereof and including any other active forms, such as racemic mixtures (D & L forms). It is expected that daily microgram amounts of ergothioneine will be effective as an antioxidant. Other antioxidants, such as selenium, are known to be effective as antioxidants at these very low levels.

Phytonutrients found in mushrooms have been the object of anticancer research. Most of this research has centered on carbohydrate-related parts of mushrooms, including their polysaccharide and beta-glucan components. In particular, these mushrooms or combinations thereof may be used to help protect against the development of breast cancer by preventing circulating levels of estrogen in the body from becoming excessive. (Excessive estrogen, or hyperestrogenemia, has been repeatedly linked to increased risk of breast cancer). This effect appears to be accomplished through inhibition of an enzyme in the body called aromatase (estrogen synthase) that is necessary for the production of estrogen. Another potential use would be for protection against UV radiation and concomitant damage to the skin as well as directly to the DNA (crosslinking and the like).

According to the invention, the compounds, ergothioneine, selenoergothioneine, and the like may be administered by any acceptable means including but not limited to the following: enteral, oral, skin patch, skin cream, liposomal carrier, nano particle carrier, etc. or any combination approach.

Another embodiment uses oxidative stress biomarkers or combinations thereof such as myloperoxidase, glutathione peroxidase (plasma and/or cellular), superoxide dismutase, glutathione, GSH, GSSH to screen for differences in levels of these compounds to signify a diagnose disease states which may be alleviated through use of mushroom or mushroom extracts. This represents very early therapy or preventative treatments for disease before traditional diagnosis which may be alleviated by early bionutrient intervention, such as with mushrooms. Administration of mushrooms may be specifically targeted based upon profiles of selenium, ergothioneine, selenoergothioneine, beta glucan, to specific markers for oxidative stress paradigm and associated disease states.

It can be seen from the foregoing that mushrooms represent a very valuable store of minerals, proteins, and the like with strong health benefits including antiviral, antioxidant, even anticancer effects that are of significant health benefit to mammals.

### SUMMARY OF THE INVENTION

The present invention relates to novel L-ergothioneine compositions derived from any source and their methods of use. The compositions and combinations of L-ergothioneine are for the treatment and prevention of neurodegeneration and treatment of radiation damage in humans and animals. Additionally, the present invention provides various method of administering these compositions and compounds to humans and animals in need thereof. Ergothioneine as used herein includes all optical isomers of ergothioneine, including D-ergothioneine, or other derivatives thereof.

The present invention provides for a pharmaceutical composition for treating a disease state or condition associated with neurodegeneration such as stroke, head trauma, subarachnoid hemorrhage, radiation damage, Alzheimer's or Parkinson's disease through administration of a therapeutically effective amount of L-ergothioneine and a carrier.

In one embodiment, the invention includes the discovery of a novel form of ergothioneine that is present in mushroom, selenoergothioneine. The compound combines selenium and L-ergothioneine to create a powerful antioxidant compound which will provide health benefits to humans. The composition has selenium that has replaced sulfur in the structure of ergothioneine forming the new compound selenoergothioneine. This new compound is analogous to selenium replacing sulfur in methoineine to form selenomethoneine.

Selenoergothioneine is synthesized by mushrooms and will have potent antioxidant benefits combining two very strong antioxidant compounds into one. This compound also represents a new form of selenium that will be more bioavailable, as is the case for selenomethioneine. It also will have unique nutritional and medicinal functions and it is likely responsible for the some of the many health benefits observed from mushrooms.

In yet another embodiment, applicants have found new cultural methods which may be used to increase natural levels of ergothioneine, to help identify mushrooms which are highest in L-ergothioneine. According to the invention an assay has been developed for identifying and quantifying L-ergothioneine in plants, particularly mushrooms. Levels of ergothioneine were measured in various genera of edible mushroom including Agaricus, Lentinula, Pleurotus and Grifola by analytical methods such as by HPLC and LC-MS. It was found that differences in the genera as well as growing conditions can impact the level of L-ergothioneine. For example altering the biomass of mycelium that forms in the substrate can lead to increased ergothioneine in the fruiting bodies. Addition of histidine to the substrates also was found to increase the ergothioneine content of the fruiting bodies. Thus applicants have identified a methodology and parameters for optimizing the best strains and environments for creating mushrooms with the highest amounts of L-ergothioneine.

Another embodiment of the invention includes the administration of mushrooms and or parts, extracts, of compounds purified therefrom in the early treatment of various pathophysiologic disease states, such as arthritis, heart disease, neurodegenerative disorders when symptoms are mild thereby avoiding the use of high dose toxic drugs. Identification and quantification of compounds present in mushrooms such as beta glucan, selenium, ergothioneine, and selenoergothioneine can be maximized for particular therapeutic value.

It has been postulated that ergothioneine exists in animals as part of an independent transporter system that includes a transporter protein molecule that could be used as part of the treatment paradigm disclosed herein. Any component of the mushroom may be used according to the invention, including one or more of the following, mycelial substrate, fruit body, gills, stapes and the like.

It has also been shown in at least one experiment that total polyphenols varied inversely with L-ergothioneine in A. bisporus mushrooms. Therefore methods were developed for maximizing ergothioneine content of mushrooms high in both of the antioxidants, polyphenols and erogothioneine. Thus methods are provided herein for maximizing the health benefits of mushrooms by increasing the antioxidants present or other valuable nutrients such as beta glucan, selenium, ergothioneine, polyphenols, or selenoergothioneine, and for identifying the strains and growing conditions associated with maximizing each.

In yet another embodiment, synthetic L-ergothioneine, a compound naturally found in mushrooms was tested and shown to have significant neuroprotective activity. Administration of L-ergothioneine correlated with viability of cortical neurons, as measured by MTT assay which measures mitochondrial dehydrogenase activity in viable cells.

Another embodiment of the invention provides for a pharmaceutical composition for treating a disease state or condition associated with neurodegeneration such as stroke, head trauma, subarachnoid hemorrhage, radiation damage, Alzheimer's or Parkinson's disease to an animal through administration of a therapeutically effective amount of L-ergothioneine and a carrier.

In another embodiment, the present invention provides for a pharmaceutical composition for prophylactic treatment of a disease state or condition associated with neurodegeneration such as stroke, head trauma, subarachnoid hemorrhage, Alzheimer's or Parkinson's disease through administration of a therapeutically effective amount of L-ergothioneine and a carrier.

In yet another embodiment, the present invention provides for a pharmaceutical composition for prophylactic treatment of a disease state or condition associated with neurodegeneration such as stroke, head trauma, subarachnoid hemorrhage, Alzheimer's or Parkinson's disease to an animal through administration of a therapeutically effective amount of L-ergothioneine and a carrier.

Another embodiment of the present invention provides for methods in prophylactic treatment and treatment of a disease state or condition associated with radiation damage and subarachnoid hemorrhage, Alzheimer's or Parkinson's disease through administration of a therapeutically effective amount of L-ergothioneine and a carrier from those suffering from said condition or disease state.

In yet another embodiment, the present invention provides for methods in administering a therapeutically effective amount of L-ergothioneine and a carrier to include enteral, oral, liposomal carrier, nano particle carrier, topical, systemic, subdermal, subcutaneous, solutions, syrups, and/or directly to the nervous system.

According to the invention, Applicants have demonstrated 1) the antioxidant properties of selenoergothioneine; 2) mushroom compositions identifying those highest in ergothioneine; and 3) neuron protective activity of L-ergothioneine.

### BRIEF DESCRIPTION OF THE DRAWINGS

FIG. 1 is the molecular structure of ergothioneine.

FIG. 2 is the molecular structure of selenoergothioneine.

FIG. 3 is HPLC chromatograms with UV-VIS absorbance units (AU) (254 nm) of mushroom extract [A] and added mushroom extract spiked with authentic ergothioineine [B].

FIG. 4 is a product ion spectra of the ion ergothioneine at m/z 230 [A] and selenoergothioneine at m/z 277 [B] in white button mushroom.

FIG. 5 is LC-MS chromatograms of mushroom sample [A] and authentic ergothioneine [B].

FIG. 6 shows the linear relationship (R²=0.86) between oxygen radical absorbance capacity (ORACₜₒₜₐₗ) expressed as micromoles Trolox equivalents per gram dry weight (μmole TE/g dw) and polyphenols expressed as milligrams gallic acid equivalents per gram dry weight (mg GAE/g dw) in cultivated mushrooms.

FIG. 7 shows at 2% low serum assay effects of T.I.1 L-ergothioneine on viability of cortical neurons obtained with the MTT method. T.I.1 addition at 1DIV. Results shown as mean and SEM of the data in % (control [0 μM] is 100%).

FIG. 8 shows at 2% low serum assay effects of R.I.1 Mangostin on viability of cortical neuron obtained with the MTT method. R.I.1 addition at 1DIV. Results shown as mean and SEM of the data in % (control [0 μM] is 100%).

FIG. 9 shows at 2% low serum assay effects of R.I.2 NTS on viability of cortical neuron obtained with the MTT method. R.I.2 addition at 1DIV. Results shown as mean and SEM of the data in % (control [0 μM] is 100%).

FIG. 10 shows at 2% low serum assay effects of R.I.3 Trolox on viability of cortical neuron obtained with the MTT method. R.I.3 addition at 1DIV. Results shown as mean and SEM of the data in % (control [0 μM] is 100%).

### DETAILED DESCRIPTION OF THE INVENTION

In accordance with the teachings of the present invention, disclosed herein are compositions, combinations and methods for the prevention and treatment of a disease state or condition associated with neurodegeneration and radiation damage. The invention relates to novel compositions and combinations containing a therapeutically effective amount of L-ergothioneine and a carrier.

L-ergothioneine is a naturally occurring antioxidant that is very stable in the body. It is synthesized in fungi and microorganisms and present in both plants and animals. Animals are unable to synthesize L-ergothioneine and must obtain it from dietary sources. It is readily absorbed and is active in most mammalian tissues, concentrating especially in the liver, where it prevents certain types of free-radical-induced damage to cell membranes and organelles. For example, exogenous L-ergothioneine has been shown to prevent lipid peroxidation by toxic compounds in the liver tissue of rats. Akanmu, D., et al., The antioxidant action of ergothioneine, Arch. of Biochemistry and Biophysics, 288 (1), 1991, pp. 10-16; Kawano, H., et al., Studies on Ergothioneine: Inhibitory effect on lipid peroxide formation in mouse liver, Chem. Pharm. Bull., 31 (5), 1983, pp. 1662-87. In studies comparing the inhibition of lipid peroxide (LPO) formation by various compounds in mouse liver, L-ergothioneine both inhibited LPO formation and enhanced the decomposition of existing LPO. Id. L-ergothioneine additionally has been shown to inhibit the damaging effects caused by the oxidation of iron-containing compounds, such as hemoglobin and myoglobin. These molecules are important in the body as carriers of oxygen, but because they contain divalent iron, they can interact with hydrogen peroxide via the Fenton reaction to produce the even more damaging hydroxyl radical. This is the mechanism by which damage occurs during so-called reperfusion injury. Because L-ergothioneine acts as a reducing agent of the ferryl-myoglobin molecule, it can protect tissues from reperfusion injury. Hanlon, D., Interaction of ergothioneine with metal ions and metalloenzymes, J. Med. Chem., 14 (11), 1971, pp. 1084-87. Although L-ergothioneine does not directly scavenge superoxide anion or hydrogen peroxide, it contributes to the control of these free radicals by participating in the activation of superoxide dismutase and glutathione peroxidase. Its protective effects on cell membranes and other organelles are of benefit in acute and chronic toxicity as well as in infectious diseases, because common pathogenic biomechanisms are active in both of these processes.

Ergothioneine and all optical isomers of ergothioneine, to include L-ergothioneine and D-ergothioneine, or other derivatives thereof, would be useful in the invention, including natural, semisynthetic, bioengineered, synthetic, extracted and combinations thereof and including any other active forms, such as racemic mixtures (D & L forms). L-ergothioneine is available commercially from Oxis International, Inc. or from dietary sources such as mushrooms. Because ergothioneine is available in nature, it is expected that daily microgram amounts will be effective as an antioxidant. Other antioxidants, such as selenium, are known to be effective as antioxidants at these very low levels.

### The Nature of Neuroprotection

Patients with injury or damage of any kind to the central (CNS) or peripheral (PNS) nervous system including the retina may benefit from these neuroprotective methods. This nervous system injury may take the form of an abrupt insult or an acute injury to the nervous system as in, for example, acute neurodegenerative disorders including, but not limited to; acute injury, hypoxia-ischemia or the combination thereof resulting in neuronal cell death or compromise. Acute injury includes, but is not limited to, Traumatic Brain Injury (TBI) including, closed, blunt or penetrating brain trauma, focal brain trauma, diffuse brain damage, spinal cord injury, intracranial or intravertebral lesions (including, but not limited to, contusion, penetration, shear, compression or laceration lesions of the spinal cord or whiplash shaken infant syndrome).

In addition, deprivation of oxygen or blood supply in general can cause acute injury as in hypoxia and/or ischemia including, but not limited to, cerebrovascular insufficiency, cerebral ischemia or cerebral infarction, including cerebral ischemia or infarctions originating from embolic occlusion and thrombosis, retinal ischemia (diabetic or otherwise), glaucoma, retinal degeneration, multiple sclerosis, toxic and ischemic optic neuropathy, reperfusion following acute ischemia, perinatal hypoxic-ischemic injury, cardiac arrest or intracranial hemorrhage of any type (including, but not limited to, epidural, subdural, subarachnoid or intracerebral hemorrhage).

Trauma or injury to tissues of the nervous system may also take the form of more chronic and progressive neurodegenerative disorders, such as those associated with progressive neuronal cell death or compromise over a period of time including, but not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases (amyotrophic lateral sclerosis), multiple sclerosis, degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease or spinocerebellar ataxia type 3 and olivopontocerebellar degenerations, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome) or prion diseases (including, but not limited to Creutzfeld-Jakob disease, Gerstmann-Strussler-Scheinker disease, Kuru disease or fatal familial insomnia).

In addition, trauma and progressive injury to the nervous system can take place in various psychiatric disorders, including but not limited to, progressive, deteriorating forms of Bipolar disorder or Schizoaffective disorder or Schizophrenia, Impulse Control disorders, Obsessive Compulsive disorder (OCD), behavioral changes in Temporal Lobe Epilepsy and personality disorders.

In one preferred embodiment the compounds of the invention would be used to provide neuroprotection in disorders involving trauma and progressive injury to the nervous system in various psychiatric disorders. These disorders would be selected form the group consisting of Schizoaffective disorder, Schizophrenia, Impulse Control disorders, Obsessive Compulsive disorder (OCD) and personality disorders.

In addition, trauma and injury make take the form of disorders associated with overt and extensive memory loss including, but not limited to, neurodegenerative disorders associated with age-related dementia, vascular dementia, diffuse white matter disease (Binswanger's disease), dementia of endocrine or metabolic origin, dementia of head trauma and diffuse brain damage, dementia pugilistica or frontal lobe dementia, including but not limited to Pick's Disease.

Other disorders associated with neuronal injury include, but are not limited to, disorders associated with chemical, toxic, infectious and radiation injury of the nervous system including the retina, injury during fetal development, prematurity at time of birth, anoxic-ischemia, injury from hepatic, glycemic, uremic, electrolyte and endocrine origin, injury of psychiatric origin (including, but not limited to, psychopathology, depression or anxiety), injury from peripheral diseases and plexopathies (including plexus palsies) or injury from neuropathy (including neuropathy selected from multifocal, sensory, motor, sensory-motor, autonomic, sensory-autonomic or demyelinating neuropathies (including, but not limited to Guillain-Barre syndrome or chronic inflammatory demyelinating polyradiculoneuropathy) or those neuropathies originating from infections, inflammation, immune disorders, drug abuse, pharmacological treatments, toxins, trauma (including, but not limited to compression, crush, laceration or segmentation traumas), metabolic disorders (including, but not limited to, endocrine or paraneoplastic), Charcot-Marie-Tooth disease (including, but not limited to, type 1a, 1 b, 2, 4a or 1-X linked), Friedreich's ataxia, metachromatic leukodystrophy, Refsum's disease, adrenomyeloneuropathy, Ataxia-telangiectasia, Djerine-Sottas (including, but not limited to, types A or B), Lambert-Eaton syndrome or disorders of the cranial nerves).

Therefore, the term “neuroprotection” as used herein shall mean inhibiting, preventing, ameliorating or reducing the severity of the dysfunction, degeneration or death of nerve cells, axons or their supporting cells in the CNS or PNS of a mammal, including a human. This includes the treatment or prophylaxis of a neurodegenerative disease; protection against excitotoxicity or ameliorating the cytotoxic effect of a compound (for example, a excitatory amino acid such as glutamate; a toxin; or a prophylactic or therapeutic compound that exerts an immediate or delayed cytotoxic side effect including but not limited to the immediate or delayed induction of apoptosis) in a patient in need thereof.

Therefore, the term “a patient in need of treatment with a neuroprotective drug (NPD)” as used herein will refer to any patient who currently has or may develop any of the above syndromes or disorders, or any disorder in which the patient's present clinical condition or prognosis could benefit from providing neuroprotection to prevent the; development, extension, worsening or increased resistance to treatment of any neurological or psychiatric disorder.

The term “antiepileptic drug” (AED) will be used interchangeably with the term “anticonvulsant agent,” and as used herein, both terms refer to an agent capable of inhibiting (e.g., preventing slowing, halting, or reversing) seizure activity or ictogenesis when the agent is administered to a subject or patient.

The term “pharmacophore” is known in the art, and, as used herein, refers to a molecular moiety capable of exerting a selected biochemical effect, e.g., inhibition of an enzyme, binding to a receptor, chelation of an ion, and the like. A selected pharmacophore can have more than one biochemical effect, e.g., can be an inhibitor of one enzyme and an agonist of a second enzyme. A therapeutic agent can include one or more pharmacophore, which can have the same or different biochemical activities.

The term “treating” or “treatment” as used herein, refers to any indicia of success in the prevention or amelioration of an injury, pathology or condition, including any objective or subjective parameter such as abatement; remission; diminishing of symptoms or making the injury, pathology, or condition more tolerable to the patient; slowing in the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluations. Accordingly, the term “treating” or “treatment” includes the administration of the compounds or agents of the present invention to provide neuroprotection. In some instances, treatment with the compounds of the present invention will do in combination with other neuroprotective compounds or AEDs to prevent, inhibit, or arrest the progression of neuronal death or damage or brain dysfunction or brain hyperexcitability.

The term “therapeutic effect” as used herein, refers to the effective provision of neuroprotection effects to prevent or minimize the death or damage or dysfunction of the cells of the patient's central or peripheral nervous system.

The term “a therapeutically effective amount” as used herein means a sufficient amount of one or more of the compounds of the invention to produce a therapeutic effect, as defined above, in a subject or patient in need of such neuroprotection treatment.

The terms “subject” or “patient” are used herein interchangeably and as used herein mean any mammal including but not limited to human beings including a human patient or subject to which the compositions of the invention can be administered. The term “mammals” include human patients and non-human primates, as well as experimental animals such as rabbits, rats, and mice, and other animals.

In some embodiments the methods of the present invention will be advantageously used to treat a patient who is not suffering or known to be suffering from a condition that is known in the art to be effectively treated with compounds or presently known neuroprotective compounds or AEDs. In these cases the decision to use the methods and compounds of the present invention would be made on the basis of determining if the patient is a “patient in need of treatment with a neuroprotective drug (NPD)”, as that term is defined above.

In some embodiments this invention provides methods of neuroprotection. In certain embodiments, these methods comprise administering a therapeutically effective amount of a compound of the invention to a patient who has not yet developed overt, clinical signs or symptoms of injury or damage to the cells of the nervous system but who may be in a high risk group for the development of neuronal damage because of injury or trauma to the nervous system or because of some known predisposition either biochemical or genetic or the finding of a verified biomarker of one or more of these disorders.

Thus, in some embodiments, the methods and compositions of the present invention are directed toward neuroprotection in a subject who is at risk of developing neuronal damage but who has not yet developed clinical evidence. This patient may simply be at “greater risk” as determined by the recognition of any factor in a subject's, or their families, medical history, physical exam or testing that is indicative of a greater than average risk for developing neuronal damage. Therefore, this determination that a patient may be at a “greater risk” by any available means can be used to determine whether the patient should be treated with the methods of the present invention.

Accordingly, in exemplary embodiments, subjects who may benefit from treatment by the methods and compounds of this invention can be identified using accepted screening methods to determine risk factors for neuronal damage. These screening methods include, for example, conventional work-ups to determine risk factors to include, for example, head trauma, either closed or penetrating, CNS infections, bacterial or viral, cerebrovascular disease including but not limited to stroke, brain tumors, brain edema, cysticercosis, porphyria, metabolic encephalopathy, drug withdrawal including but not limited to sedative-hypnotic or alcohol withdrawal, abnormal perinatal history including anoxia at birth or birth injury of any kind, cerebral palsy, learning disabilities, hyperactivity, history of febrile convulsions as a child, history of status epilepticus, family history of epilepsy or any a seizure related disorder, inflammatory disease of the brain including lupis, drug intoxication either direct or by placental transfer, including but not limited to cocaine poisoning, parental consanguinity, and treatment with medications that are toxic to the nervous system including psychotropic medications.

The determination of which patients may benefit from treatment with an NPD in patients who have no clinical signs or symptoms may be based on a variety of “surrogate markers” or “biomarkers.”

As used herein, the terms “surrogate marker” and “biomarker” are used interchangeably and refer to any anatomical, biochemical, structural, electrical, genetic or chemical indicator or marker that can be reliably correlated with the present existence or future development of neuronal damage. In some instances, brain-imaging techniques, such as computer tomography (CT), magnetic resonance imaging (MRI) or positron emission tomography (PET), can be used to determine whether a subject is at risk for neuronal damage.

Suitable biomarkers for the methods of this invention include, but are not limited to, the determination by MRI, CT or other imaging techniques, of sclerosis, atrophy or volume loss in the hippocampus or overt mesial temporal sclerosis (MTS) or similar relevant anatomical pathology; the detection in the patient's blood, serum or tissues of a molecular species such as a protein or other biochemical biomarker, e.g., elevated levels of ciliary neurotrophic factor (CNTF) or elevated serum levels of a neuronal degradation product; or other evidence from surrogate markers or biomarkers that the patient is in need of treatment with a neuroprotective drug.

It is expected that many more such biomarkers utilizing a wide variety of detection techniques will be developed in the future. It is intended that any such marker or indicator of the existence or possible future development of neuronal damage, as the latter term is used herein, may be used in the methods of this invention for determining the need for treatment with the compounds and methods of this invention.

A determination that a subject has, or may be at risk for developing, neuronal damage would also include, for example, a medical evaluation that includes a thorough history, a physical examination, and a series of relevant bloods tests. It can also include an electroencephalogram (EEG), CT, MRI or PET scan. A determination of an increased risk of developing neuronal damage or injury may also be made by means of genetic testing, including gene expression profiling or proteomic techniques. (See, Schmidt, D. Rogawski, M. A. Epilepsy Research 50; 71-78 (2002), and Loscher, W, Schmidt D. Epilepsy Research 50; 3-16 (2002)).

For psychiatric disorders that may be stabilized or improved by a neuroprotective drug, e.g., Bipolar Disorder, Schizoaffective disorder, Schizophrenia, Impulse Control Disorders, etc. the above tests may also include a present state exam and a detailed history of the course of the patients symptoms such as mood disorder symptoms and psychotic symptoms over time and in relation to other treatments the patient may have received over time, e.g., a life chart. These and other specialized and routine methods allow the clinician to select patients in need of therapy using the methods and formulations of this invention.

In some embodiments of the present invention compounds suitable for use in the practice of this invention will be administered either singly or concomitantly with at least one or more other compounds or therapeutic agents, e.g., with other neuroprotective drugs or antiepileptic drugs, anticonvulsant drugs. In these embodiments, the present invention provides methods to treat or prevent neuronal injury in a patient. The method includes the step of administering to a patient in need of treatment an effective amount of one of the compounds disclosed herein in combination with an effective amount of one or more other compounds or therapeutic agents that have the ability to provide neuroprotection or to treat or prevent seizures or epileptogenesis or the ability to augment the neuroprotective effects of the compounds of the invention.

“Concomitant administration” or “combination administration” of a compound, therapeutic agent or known drug with a compound of the present invention means administration of the drug and the one or more compounds at such time that both the known drug and the compound will have a therapeutic effect. In some cases this therapeutic effect will be synergistic. Such concomitant administration can involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compounds of the present invention.

In addition, in some embodiments, the compounds of this invention will be used, either alone or in combination with each other or in combination with one or more other therapeutic medications as described above, or their salts or esters, for manufacturing a medicament for the purpose of providing neuroprotection to a patient or subject in need thereof.

In general, the compounds of the present invention can be administered as pharmaceutical compositions by any method known in the art for administering therapeutic drugs including oral, buccal, topical, systemic (e.g., transdermal, intranasal, or by suppository), or parenteral (e.g., intramuscular, subcutaneous, or intravenous injection.) Administration of the compounds directly to the nervous system can include, for example, administration to intracerebral, intraventricular, intacerebroventricular, intrathecal, intracisternal, intraspinal or peri-spinal routes of administration by delivery via intracranial or intravertebral needles or catheters with or without pump devices.

In addition, in the case of diseases or disorders of the eye including, but not limited to, retinal ischemia (diabetic or otherwise), glaucoma, retinal degeneration, macular degeneration, multiple sclerosis, toxic and ischemic optic neuropathy the compounds of the present invention, including combinations of compounds, can be administered by means of direct exogenous application to the eye, i.e., to the sclera or otherwise, e.g., eye drops or by ocular implant or other slow delivery device including microspheres including by direct injection into the vitreous humor etc.

Compositions can take the form of tablets, pills, capsules, semisolids, powders, sustained release formulations, solutions, suspensions, emulsions, syrups, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, can be found in such standard references as Alfonso AR: Reminqton's Pharmaceutical Sciences, 17th ed., Mack Publishing Company, Easton Pa., 1985, the disclosure of which is incorporated herein by reference in its entirety and for all purposes. Suitable liquid carriers, especially for injectable solutions, include water, aqueous saline solution, aqueous dextrose solution, and glycols.

Pharmaceutical formulations for oral administration can be formulated using pharmaceutically acceptable carriers well known in the art in dosages suitable for oral administration. Such carriers enable the pharmaceutical formulations to be formulated in unit dosage forms as tablets, pills, powder, dragees, capsules, liquids, lozenges, gels, syrups, slurries, suspensions, etc. suitable for ingestion by the patient.

Formulations suitable for oral administration can consist of (a) liquid solutions, such as an effective amount of the pharmaceutical formulation suspended in a diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions.

The compounds of the present invention can also be administered in the form of suppositories for rectal administration of the drug. These formulations can be prepared by mixing the drug with a suitable non-irritating excipient that is solid at ordinary temperatures but liquid at the rectal temperatures and will therefore melt in the rectum to release the drug. Such materials are cocoa butter and polyethylene glycols.

The compounds of the present invention can also be administered by intranasal, intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

The compounds of the present invention can be delivered transdermally, by a topical route, formulated as applicator sticks, solutions, suspensions, emulsions, gels, creams, ointments, pastes, jellies, paints, powders, and aerosols.

Encapsulating materials can also be employed with the compounds of the present invention and the term “composition” can include the active ingredient in combination with an encapsulating material as a formulation, with or without other carriers. For example, the compounds of the present invention can also be delivered as microspheres for slow release in the body. In one embodiment, microspheres can be administered via intradermal injection of drug (e.g., mifepristone)-containing microspheres, which slowly release subcutaneously (see Rao, J. Biomater Sci. Polym. Ed. 7:623-645, 1995; as biodegradable and injectable gel formulations (see, e.g., Gao, Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months. Cachets can also be used in the delivery of the compounds of the present invention.

Methods of formulating pharmaceutical compositions have been described in numerous publications such as Pharmaceutical Dosage Forms: Tablets. Second Edition. Revised and Expanded. Volumes 1-3, edited by Lieberman et al; Pharmaceutical Dosage Forms: Parenteral Medications. Volumes 1-2, edited by Avis et al; and Pharmaceutical Dosage Forms: Disperse Systems. Volumes 1-2, edited by Lieberman et al; published by Marcel Dekker, Inc, the disclosure of which are herein incorporated by reference in their entireties and for all purposes.

The pharmaceutical compositions are generally formulated as sterile, substantially isotonic and in full compliance with all Good Manufacturing Practice (GMP) regulations of the U.S. Food and Drug Administration

The methods of this invention also provide for kits for use in providing neuroprotection. After a pharmaceutical composition comprising one or more compounds of this invention, with the possible addition of one or more other compounds of therapeutic benefit, has been formulated in a suitable carrier, it can be placed in an appropriate container and labeled for providing neuroprotection. Additionally, another pharmaceutical comprising at least one other therapeutic agent useful in the provide neuroprotection, treatment of epileptogenesis, epilepsy or another disorder or condition associated with neuronal injury can be placed in the container as well and labeled for treatment of the indicated disease. Such labeling can include, for example, instructions concerning the amount, frequency and method of administration of each pharmaceutical.

Although the foregoing invention has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. The following example is provided to illustrate specific aspects of the invention and are not meant to be limitations.

### Administration and Dose Ranges

The compositions and combinations of the present invention can be administered by a variety of routes including, but not limited to: orally, parentally, transdermally, sublingually, intravenously, intramuscularly, rectally and subcutaneously. Preferred daily doses for cach of the compounds are as follows. As would be apparent to a person of ordinary skill in the art, these dose ranges are approximations:

L-ergothioneine

Total dose range: about 5 .mu.g—about 25 grams

Preferred small animal dose range: about 5 .mu.g—about 5 grams

Preferred human dose range: about 25 .mu.g—about 10 grams

Preferred large animal dose range: about 100 .mu.g—about 25 grams

Alternatively, the daily per kilogram dose range of L-ergothionine for all species is: about 2 .mu.g/kg—about 250 mg/kg.

The daily doses recited above for all compounds may be given in a single dose or divided doses, to be administered, for example, twice-a-day, three-times a day, or four-times-a-day. Therefore, the range for a single dose of the components of the invention is as follows:

L-ergothioneine

Total single dose range: about 1.25 .mu.g—about 25 grams

Preferred small animal single dose range: about 1.25 .mu.g—about 5 grams

Preferred human single dose range: about 6.25 .mu.g—about 10 grams

Preferred large animal single dose range: about 25 .mu.g—about 25 grams

Alternatively, the per kilogram single dose range for all species is: about 0.5..mu.g/kg—about 250 mg/kg.

Moreover, the dose may be administered in various combinations in which the components may be present in a single dosage form or in more than one dosage form. For example, the combinations of the present invention may be administered in a single daily dosage form in which all components are present, e.g., in a single capsule or tablet. The doses may also be administered in combinations of more than one dosage form in which each dosage form contains at least one component or in which two or more components are combined into a single dosage form. These combinations may be provided in kits or blister packs, in which more than one dosage form of the various components are provided in the same package or container, for co-administration to a human or animal.

### EXAMPLE 1

Analysis of A. bisporus mushrooms was carried out using an HPLC 600 E system controller. Separation was carried out on two Econosphere C18 columns (Alltech Associates, Deerfield, Ill.) with each column being 250 mm×4.6 mm, five-micron particle size that were connected in tandem. The degassed (ultrapure nitrogen) isocratic mobile phase was 50 mM sodium phosphate in water with 3% acetonitrile and 0.1% triethylamine adjusted to a pH of 7.3 with a flow rate of 1 mL/min. An UV-VIS 490E detector (Waters Corp., Milford, Conn.) equipped with a wavelength of 254 nm was employed. The injection volume was 10 μL with the columns temperature being ambient. Ergothioneine was quantified by monitoring absorbance at 254 nm and comparing the peak area of the sample to peak areas obtained from different concentrations of the authentic standard ergothioneine (Strip chart recorder 3392A integrator; Hewlett Packard, Palto Alto, Calif.).

Ergothioneine “spiked” samples were prepared by dissolving 0.2 mg of authentic ergothioneine in 1.0 mL of water and added to the ethanolic extraction medium to yield the same volume as the control mushroom samples (20 mL). The “spiked” samples were then completed and analyzed as previously described. Using the previously described HLPC methodology, comparisons of the peak areas were made with samples to which no ergothioneine has been added.

To confirm the identity of the analyte, LC-MS was used. Mushroom samples were analyzed by an Shimadzu HPLC (Shimadzu, Columbia, Md.) equipped with a pump (LC-10ATvp), degasser (DGU-14A), low-pressure mixture (FCV-10ALVP), an autosampler (SIL-19vp), and two 250 mm×4.6 mm, five-micron particle size Econosphere C18 columns (Alltech Associates, Deerfield, Ill.) connected in tandem interfaced to a Waters ZMD 2000 Mass Spectrometer (Waters Corp., Milford, Mass.). HPLC conditions were as follows: injection volume was 10 μL, columns temperatures were ambient, isocratic mobile system (3% acetonitrile and 0.1% acetic acid in water) and the column flow rate was 1.0 mL/min. The effluent was split 10:1 post column (zero-dead volume Tsplitter; Supelco, Bellefonte, Pa.) with 1 part (100 μL/min) directed to the MS for detection and the other to waste. The MS conditions were as follows: electrospray ionization in positive ion mode, capillary voltage (3.0 Kvolts); scan range (105 to 500 Da); source temperature (100° C.); probe temperature (250° C.).

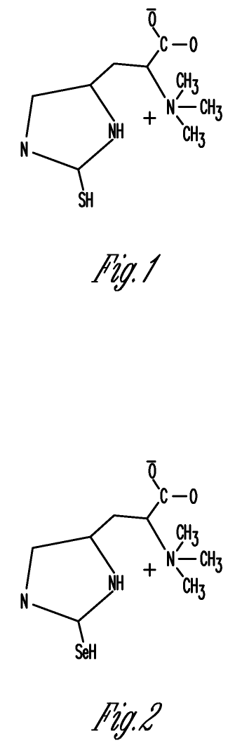

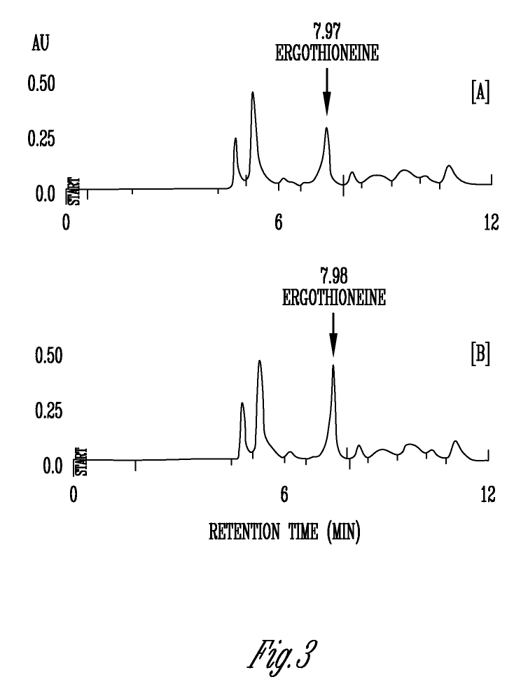

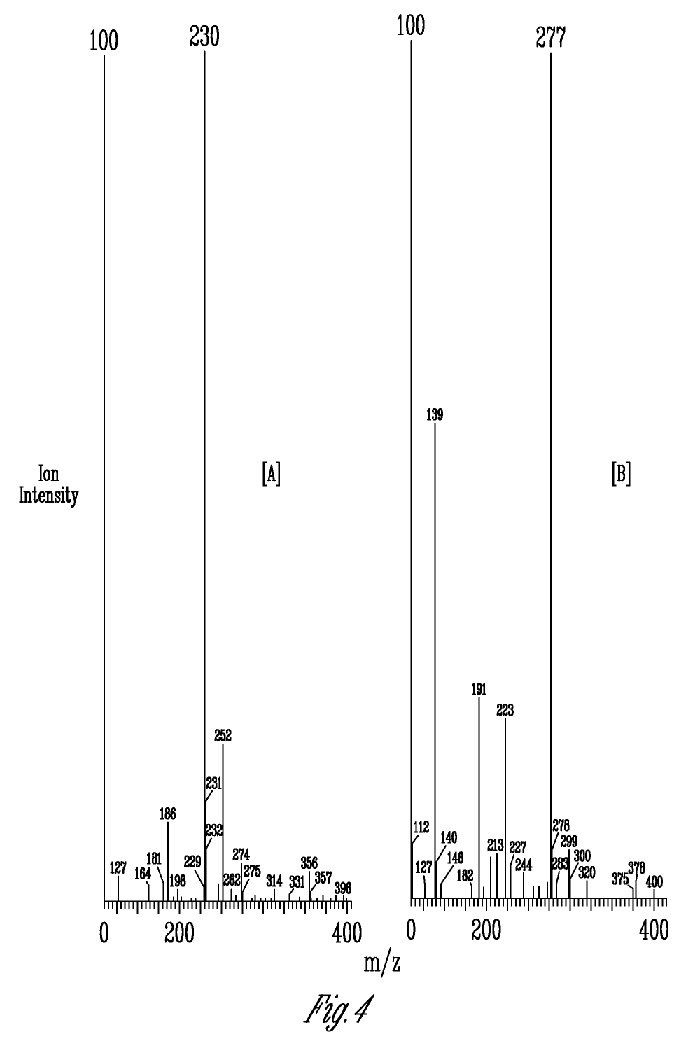

The results of the “spiked” samples demonstrated that added ergothioneine could be accurately recovered by this procedure since recoveries were ≧95%. (FIG. 3). The first step of this work involved the characterization of the mass spectral properties of ergothioneine. FIG. 3 shows the MS product ion scan spectra of ergothioneine (m/z 230) in a white button mushroom sample. The analyte of interest was identified by comparison of its retention time and MS spectrum in full scan mode with that of a reference compound and a mushroom sample. Retention times and MS spectra of the mushroom sample were identical to those of the standard. Initially the mobile phase consisted of 3% acetonitrile in water with the pH adjusted to 7.3 by ammonium hydroxide. The mass spectra showed a prominent molecular ion at a m/z of 252 (data not shown). It is believed that a sodium ion attached to the structure of ergothioneine, which resulted in a m/z of 252 (refer to FIG. 1). With a sodium ion bound to the carboxyl group, a M+Na ion was formed. The acidity of the mobile phase was adjusted by adding 0.1 % (v/v) acetic acid in order to protonate the carboxyl group (remove Na). With this slight modification the background noise increased and the analyte is not as predominate; however, the sodium was removed which resulted in the reported m/z of 230.

Subsequent examination of the mass spectra revealed a m/z of 277 (FIG. 4[B] which suggests that selenium had replaced the sulfur in ergothioneine resulting in seleno-ergothioneine with a m/z of 277 (FIG. 4). It appears competitive uptake occurs between the sulfur and the selenium that is naturally present in the compost and casing ingredients as occurs in the formation of selenoergothioneine in mushrooms. The data suggests that this amino acid derivative synthesized by mushrooms may have unique bioactive properties and therefore may be a novel component in mushrooms that is a potential antioxidant with unique nutritional or medicinal functions.

### EXAMPLE 2

Various mushrooms were collected and analyzed for ergothioneine content (Table 1). The A. bisporus mushrooms were grown using the standard tray system (Hartman, 1998) under standard, controlled conditions that are representative of growth conditions at mushroom farms across the country (Wuest et al., 1986). The mushrooms were harvested in optimum maturation stage with the caps closed and 2.0 to 2.5 inches in diameter. Second breaks from the A. bisporus crops were tested. Samples from the A. bisporus included in this survey were brown (crimini) mushrooms, portabella mushrooms (mature, brown mushrooms with exposed hymenia), and the common white button mushrooms. Also, in another experiment a crop of both white and brown strains of A. bisporus mushrooms were grown simultaneously using the same growing conditions and on the same compost. This experiment was conducted in order to determine if strain or stage of maturity of fruiting bodies at harvest has an effect on ergothioneine levels in mushrooms. The growing conditions of the specialty mushrooms were those currently employed at mushroom farms across the country; thus, the mushrooms analyzed would be comparable to those normally available to consumers. Mushrooms for analysis were harvested on the peak production day.

Harvested mushrooms were expediently transferred by vehicle from the growing facilities to the laboratory. Immediately following this and after random sampling from each genera of the mushroom crop, the mushrooms were cleaned, sliced and stored in a walk-in cooler at 0° C. for 24 hours. The mushrooms were then freeze-dried (Model 15 SRC-X; Virtis Genesis Co., Inc., Gardiner, N.Y.), ground to a fine powder and sieved through a size 16 mesh screen. The mushroom powder was collected in sterile sample bags (Fisher Scientific, Pittsburgh, Pa.) and stored in the dark at room temperature in desiccators over CaSO₄. One gram of freeze-dried mushroom powder was dispersed in 20 mL of cold ethanolic extraction medium (10 mM DTT, 100 μM betaine and 100 μM MMI in 70% ethanol) and mixed using a small blender (Model LB10 and 37 mL stainless steel container; Waring Laboratory, Torrington, Conn.). A 1.0% ethanolic solution (4 mL) of SDS was added and gently mixed. Centrifugation (Allegra 6R; Beckman Coulter, Fullerton, Calif.) was completed for 20 minutes at 4000 rpm in order to remove insoluble material. One mL of the vortexed supernatant was evaporated to dryness under a stream of ultrapure nitrogen gas. The residue was then resuspended in 0.5 mL of water (adjusted to a pH of 7.3) and microcentrifuged (Scientific model V; VWR, Bristol, Conn.) for one minute at 10,000 rpm. The supernatant was filtered through a 0.45 micron filter (Alltech Associates, Deerfield, Ill.) prior to injection into the HPLC.

Analysis was carried out using an HPLC 600E system controller (Waters Corp., Milford, Conn.). Separation was carried out on two Econosphere C18 columns (Alltech Associates, Deerfield, Ill.) with each column being 250 mm×4.6 mm, five-micron particle size that were connected in tandem. The degassed (ultrapure nitrogen) isocratic mobile phase was 50 mM sodium phosphate in water with 3% acetonitrile and 0.1% triethylamine adjusted to a pH of 7.3 with a flow rate of 1 mL per minute. An UV-VIS 490E detector (Waters Corp., Milford, Conn.) equipped with a wavelength of 254 nm was employed. The injection volume was 10 μL with the columns temperature being ambient. Ergothioneine was quantified by monitoring absorbance at 254 nm and comparing the peak area of the sample to peak areas obtained from different concentrations of the authentic standard ergothioneine (Strip chart recorder 3392A integrator; Hewlett Packard, Palto Alto, Calif.).

Ergothioneine “spiked” samples were prepared by dissolving 0.2 mg of authentic ergothioneine in 1.0 mL of water and added to the ethanolic extraction medium to yield the same volume as the control mushroom samples (20 mL). The “spiked” samples were then completed and analyzed as previously described. Using the previously described HPLC methodology, comparisons of the peak areas were made with samples to which no ergothioneine has been added. LC-MS. To confirm the identity of the analyte, LC-MS was used. Mushroom samples were analyzed by an Shimadzu HPLC (Shimadzu, Columbia, Md.) equipped with a pump (LC-10ATvp), degasser (DGU-14A), low-pressure mixture (FCV-10ALVP), an autosampler (SIL-19vp), and two 250 mm×4.6 mm, five-micron particle size Econosphere C18 columns (Alltech Associates, Deerfield, Ill.) connected in tandem interfaced to a Waters ZMD 2000 Mass Spectrometer (Waters Corp., Milford, Mass.). HPLC conditions were as follows: injection volume was 10 μL, columns temperatures were ambient, isocratic mobile system (3% acetonitrile and 0.1% acetic acid in water) and the column flow rate was 1.0 mL/min. The effluent was split 10:1 post column (zero-dead volume Tsplitter; Supelco, Bellefonte, Pa.) with 1 part (100 μL/min) directed to the MS for detection and the other to waste. The MS conditions were as follows: electrospray ionization in positive ion mode, capillary voltage (3.0 Kvolts); scan range (105 to 500 Da); source temperature (100° C.); probe temperature (250° C.).

A typical HPLC chromatogram of a mushroom extract and an extract spiked with ergothioneine illustrate the co-elution of the natural ergothioneine and the added standard (FIG. 3). The results of the “spiked” samples demonstrated that added ergothioneine could be accurately recovered by this procedure since recoveries were ≧95%.

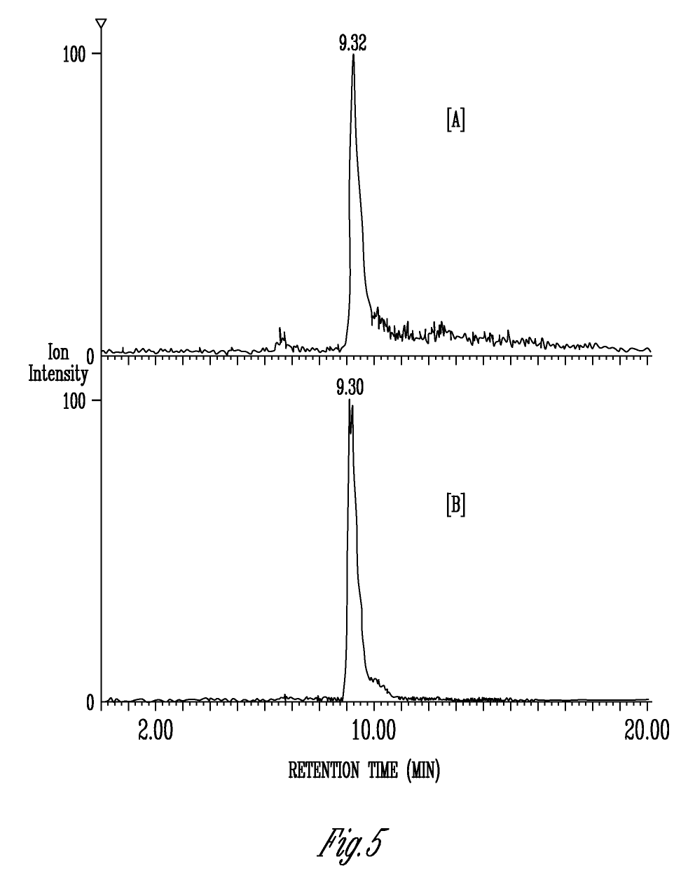

The first step of this work involved the characterization of the mass spectral properties of ergothioneine. FIG. 4 shows the MS product ion scan spectra of ergothioneine (m/z 230) in a white button mushroom sample. The analyte of interest was identified by comparison of its retention time and MS spectrum in full scan mode with that of a reference compound and a mushroom sample. Retention times and MS spectra of the mushroom sample were identical to those of the standard. Initially the mobile phase consisted of 3% acetonitrile in water with the pH adjusted to 7.3 by ammonium hydroxide. The mass spectra showed a prominent molecular ion at a m/z of 252. It is believed that a sodium ion attached to the structure of ergothioneine, which resulted in an m/z of 252 (refer to FIG. 1). With a sodium ion bound to the carboxyl group, a M+Na ion was formed. The acidity of the mobile phase was adjusted by adding 0.1% (v/v) acetic acid in order to protonate the carboxyl group (remove Na). With this slight modification the background noise increased and the analyte is not as predominate; however, the sodium was removed which resulted in the reported m/z of 230. FIG. 5 shows a comparison of the MS total ion chromatogram of the reference and the mushroom sample (background noise was subtracted from spectra).

Prior research has shown that there is a linear uptake of selenium by A. bisporus depending on the concentration of selenium in the compost; therefore, natural selenium levels affect the levels of the selenium taken up by A. bisporus (Werner and Beelman, 2002). Further examination of the mass spectra revealed a m/z of 277 (FIG. 4). This suggested that selenium may be the sulfur equivalent of ergothioneine resulting in a m/z of 277. There may be some competitive uptake within the structure of ergothioneine between the sulfur and the selenium that is naturally present in the compost and casing ingredients. If this is the case, selenoergothioneine could be further investigated as an amino acid derivative synthesized by mushrooms that may be a unique biologically-active component in mushrooms that is a potential antioxidant, as well as a new organic form of selenium.

A survey of the mushrooms tested (Agaricus bisporus (white, crimini and portabella), Lentinula edodes (shiitake), Pleurotus ostreatus (oyster), Pleurotus eryngii (king oyster) and Grifola frondosa (maitake)) indicated that all of the mushrooms contained ergothioneine but in varying amounts (Table 2). Ergothioneine present in the mushrooms ranged from 0.4-2.0 mg/g dry weight (dw). There was no significant difference found within the different A. bisporus mushrooms. However, a trend was evident where the common white button mushroom contained the least ergothioneine and portabellas contained the highest value within the genera of A. bisporus. The other mushrooms tested (shiitake, oyster, king oyster and maitake) all contained greater amounts of ergothioneine but there were no significant differences found between these specialty mushrooms. Shiitake and oyster contained the highest level of ergothioneine at approximately 2.0 mg/g dw.

A separate study was conducted to determine if different strains and maturities of A. bisporus mushrooms would produce varying amounts of ergothioneine within the fruiting bodies. Both white and brown strains of A. bisporus were grown at the MRC under the same conditions and on the same substrate (compost). The white strain was harvested as normal white button mushrooms and the brown strains as crimini (buttons) and fully mature (portabellas). There were significant differences found between the white button and the crimini and the portabella (Table 3). The white button contained 0.47±0.03 mg/g dw of ergothioneine versus the crimini and the portabella which contained 0.83±0.01 and 0.72±0.01 mg/g dw of ergothioneine, respectively. Based on the amount of ergothioneine found in the white button and on a fresh weight basis, a serving of these mushrooms (85 g) would provide 2.8 mg of ergothioneine. Also a serving of crimini and portabella would provide 4.9 and 4.3 mg of ergothioneine, respectively. These results demonstrated that there was a significant difference between the brown and white strains of A. bisporus in the level of ergothioneine produced by the mushroom. The relatively small SD was likely due to the analyzed samples originating from one crop. In the results previously discussed, a significant difference was not found between these different types of A. bisporus Mushrooms. One explanation for this could be due to environmental variation that is normally observed when comparing crops of the same type of mushroom, which tends to result in a larger SD.

Overall, the results of this study suggest that differences in strain along with growing conditions can affect the level of ergothioneine produced in the mushrooms.

TABLE 1
List of fungi surveyed for ergothioneine content.

| Mushrooms species | Sample type | Source |
|---|---|---|
| Agaricus bisporus | White button | Penn State University |
| | (all crops) | |
| | Brown mushroom | Penn State University |
| | (crimini) | |
| | Portabella | Penn State University |
| Lentinula edodes | Basidioma | Modern Mushroom Farm, Inc. |
| Grifola frondosa | Basidioma | Modern Mushroom Farm Inc. |
| | | Penn State University |
| Pleurotus osteratus | Basidioma | Modern Mushroom Farm Inc. |
| Pleurotus eryngii | Basidioma | Modern Mushroom Farm Inc. |

TABLE 2
Concentration (mean mg/g dry weight (dw) ± standard
deviation) of ergothioneine in various mushrooms.

| Sample type | mg/g dw of ergothioneine | n^{c} | c^{d} |
|---|---|---|---|
| White button | 0.41 ± 0.18^{a} | 12 | 4 |
| Crimini | 0.68 ± 0.11^{a} | 6 | 6 |
| Portabell | 0.68 ± 0.04^{a} | 5 | 5 |
| King Oyster | 1.72 ± 0.10^{b} | 3 | 1 |
| Maitake | 1.84 ± 0.76^{b} | 9 | 3 |
| Oyster | 2.01 ± 0.05^{b} | 3 | 1 |
| Shiitake | 2.09 ± 0.21^{b} | 4 | 1 |

Standard deviation followed by different letters differ significantly (p = 0.05, Tukey's method).
^{c}Sample numbr tested.
^{d}Crop number tested.

TABLE 3
Concentration (mean mg/g dry weight (dw) ± standard deviation;
mg/85 g serving size fresh weight) of ergothioneine in one
crop of Agaricus bisporus grown at Pennsyvania State University (PSU).

| Sample type (A. bisporus) | Mg/g dw of LE | n^{d} | mg/85 g fw | Source |
|---|---|---|---|---|
| White buttom | 0.47 ± 0.03^{a} | 3 | 2.8 | PSU |
| Crimini | 0.83 ± 0.01^{c} | 3 | 4.9 | PSU |
| Portabell | 0.72 ± 0.01^{b} | 3 | 4.3 | PSU |

Means followed by different letters differ significantly (p = 0.05, Tukey's method).
^{d}Sample numbr tested.

### EXAMPLE 3

Various mushrooms were collected and analyzed to measure antioxidant capacity. The most commonly consumed mushrooms in the United States, Agaricus bisporus (white, crimini and portabella), and the specialty strains, Lentinula edodes (shiitake), Pleurotus osteratus (oyster) and Grifola frondosa (maitake) were selected to determine the antioxidant capacity (radical scavenging and chelating ability) using multiple assays, including the oxygen radical absorbance capacity (ORAC_{ROO}., both hydrophilic and lipophilic), hydroxyl radical averting capacity (HORAC), peroxynitrite radical averting capacity (NORAC), and super-oxide radical averting capacity (SORAC). ERG was quantified in each of these mushrooms in order to correlate this data with the antioxidant capacity assays as an indicator of whether ERG is contributing to the antioxidant capacity of the mushrooms tested. In addition, FCR was used to quantify the total phenolics (TP) of the same mushrooms in order to evaluate their contribution to the antioxidant capacity.

Ergothioneine standard, ethanol (HPLC grade), acetonitrile (HPLC grade), diethiothreitol (DTT), betaine, 2-mercapto-1-methyl imidazole (MMI), sodium dodecylsulfate (SDS), sodium phosphate, triethylamine, Folin-Ciocalteu reagent (2.0M), hydrogen peroxide, gallic acid, caffeic acid, ethanol, xanthine oxidase, xanthine, superoxide dismutase from bovine erythrocytes, dihydrorhodamine (DHR-123), 3-morpholinosydnonimine hydrochloride (SIN-1) and sodium carbonate were purchased from Sigma Chemical Co. (St. Louis, Mo.). 2,2′-Azobis(2-amidinopropane) dihydrochloride (AAPH) was purchased from Wako Chemicals USA (Richmond, Va.). 6-Hydroxy-2,5,7,8-tetramethylchroman2-carboxylic acid (Trolox), Cobalt(II) fluoride tetrahydrate, picolinic acid (PA) and fluorescein disodium (FI) were obtained from Aldrich (Milwaukee, Wis.). Randomly methylated β-cyclodextrin (RMCD) was obtained from Cyclodextrin Technologies Development Inc. (High Springs, Fla.). Hydroethidine fluorescent stain was purchased from Polysciences, Inc. (Warrington, Pa.). ORAC, HORAC, SOAC and NORAC analysis was performed using a Precision 2000 eight channel liquid landling system and Syndergy HT microplate UV-VIS and fluorescence reader (Bio-tek. Inc., Winooski, Vt.). Total phenolics (TP) were analyzed on an UV-VIS spectrophotometer at a wavelength of 765 nm (Thermo Spectronic, Rochester, N.Y.). Quantification of ERG was completed by an HPLC 600E system controller (Waters Corp., Milford, Conn.) and a UV-VIS 490E detector (Waters Corp., Milford, Conn.).

A list of the fungi analyzed is shown in Table 4. The A. bisporus mushrooms were grown using the standard tray system (27) under standard, controlled conditions that are representative of growth conditions at mushroom farms across the country. The mushrooms are harvested in optimum maturation stage with the caps closed and 2.0 to 2.5 inches in diameter. Mushrooms from the second break of the A. bisporus crops were tested. Samples from the A. bisporus included in this survey were brown (crimini) mushrooms, portabella mushrooms (mature, brown mushrooms with exposed hymenia), and the common white button mushrooms. The growing conditions of the specialty mushrooms were those currently employed at mushroom farms across the country; thus, the mushrooms anayzed would be comparable to those normally available to consumers. Mushrooms for analysis were harvested on the peak production day.

Following random sampling from each genera of the mushroom crop, the mushrooms were cleaned, sliced and stored in a walk-in cooler at 0° C. for 24 hours. The mushrooms were then freeze-dried (Model 15 SRC-X; Virtis Genesis Co., Inc., Gardiner, N.Y.), ground to a fine powder and sieved through a size 16 mesh screen.

Total phenolics (TP) concentrations were measured using FCR. The ethanolic extracts were used for analyzing their phenolic compounds following a modified method of Fu and co-workers (28). Five grams of the freeze-dried mushroom poweder was added to 60 ml of 80% ethanol and heated to 60 C for one hour using a water bath (Precision Scientific Co., Chicago, Ill.). The sample was filtered after one hour and the procedure was repeated two additional times. After a total of three hours, the extract was filtered, combined with the previous extracts and diluted with 80% ethanol to a final volume of 200 ml, which was then well blended (Model LB10 with 250 ml stainless steel container: Waring laboratory, Torrington, Conn.) for 30 seconds. One ml of the ethanolic extract was added to four mls of the FCR, which was diluted with distilled water (1:10). After three minutes, 5 ml of a 7.5% aqueous sodium carbonate solutions was added and then after 30 minutes, absorbance was measured by an UV-VIX spectrophotometer at a wavelength of 765 nm (Thermo Spectronic, Rochester, N.Y.). Gallic acid was used as the standard in order to create a calibration curve by plotting absorbance versus concentration. TP content was standardized against gallic acid and the data was expressed as milligrams of gallic acid equivalents (GAE) per gram of dry weight (mg GAE/g dw). The linearity range for this assay was determined as 0.025-0.3 mg/ml GAE (R²=0.9964), yielding an average absorbance range of 0.15-2.7 AU. Each sample was extracted in triplicate with from one crop for each genera of mushroom analyzed.

The method used to quantify ERG in the mushrooms was carried out using an HPLC with separation carried out on two Econosphere C18 columns (Alltech Associates, Deerfield, Ill.) with each column being 250 mm×4.6 mm, five-micron particle size connected in tandem. The isocratic mobile phase was 50 mM sodium phosphate in water with 3% acetonitrile and 0.1% triethylamine adjusted to a pH of 7.3 with a flow rate of 1 mL per minute. An UV-VIS detector equipped with a wavelength of 254 nm was employed. The injection volume was 10 μL with the columns temperature being ambient. ERG was quantified by monitoring absorbance at 254 nm and comparing the peak area of the sample to peak areas obtained from different concentrations of the authentic standard. All data was expressed as milligrams of ERG per gram of dry weight (mg ERG/g dw). Triplicate extractions were performed and used for ERG analysis from one crop for each of mushroom genera tested.

The lipophilic ORAC_{FL} assay (ORACₗᵢₚₒ) was based on a previous reported method. Hydrophilic ORAC_{FL} assay (ORAC_{hydro}) was based on a previous reported method. All data was expressed as micromoles of Trolox equivalents (TE) per gram of dry weight (μmol TE/g dw). The freeze-dried forms of the mushrooms were used for testing and the data was reported as dry weight. When reporting amount per serving, the data was converted to a fresh weight basis according to the dry weight of the fresh mushroom samples. For each analysis, triplicate extractions were performed from one crop and used for analysis. ORACₜₒₜₐₗ was determined by calculating the sum of ORACₗᵢₚₒ and ORAC_{hydro} results.

The HORAC assay was based on a previously reported method, however caffeic acid (CA) was used as the standard due to CA providing a wider linear range as compared to gallic acid. All data was expressed as micromoles of caffeic acid equivalents (CAE) equivalents per gram of dry weight (μmol CAE/g dw). The freeze-dried forms of the mushrooms were used for testing and the data was reported as dry weight. When reporting amount per serving, the data was converted to a fresh weight basis according to the dry weight of the fresh mushroom samples. For each analysis, triplicate extractions were performed from one crop and used for analysis.

The NORAC assay was modified on a previously reported method. All data was expressed as micromoles of Trolox equivalents (TE) equivalents per gram of dry weight (μmol of TE/g dw). The freeze-dried forms of the mushrooms were used for testing and then the data was converted to a FW basis according to the dry weight of the fresh mushroom samples, which were obtained from one crop. For each analysis, triplicate extractions were performed and used for analysis.

SORAC assays were based on the previously described method by Zhao and coworkers. Simply, hydroethidine was used as a probe in measuring O₂⁻ scavenging capacity. Nonfluorescent hydroethidine was oxidized by O₂⁻ generated by the mixture of xanthine and xanthine oxidase to form a species of unknown structure that exhibit strong fluorescence signal at 586 nm. Addition of SOD inhibits the hydroethidine oxidation. All data was expressed as superoxide equivalents per gram of dry weight (kunitSOD eq/g dw). The freeze-dried forms of the mushrooms were used for testing and the data was reported as dry weight. When reporting amount per serving, the data was converted to a fresh weight basis according to the dry weight of the fresh mushroom samples. For each analysis, triplicate extractions from one crop were performed and used for analysis.

Using multiple assays, such as ORAC, NORAC, SORAC and HORAC, along with the quantification of TP provides evidence of the antioxidant capacity within the food material able to scavenge various biological significant free radicals.

ERG is present in human tissues at concentrations up to 1-2 mM. In this current study ERG concentration of the mushrooms ranged from 0.21-2.6 mg/g dw (Table 5). There was no significant difference found within A. bisporus mushrooms; however a trend was evident where the common white button mushroom contained the least ERG and portabellas contained the highest value. The specialty strains tested all contained greater amounts of ERG as compared to A. bisporus. A significant difference was found between all of the specialty mushrooms. Among the specialty mushrooms, maitake contained the least ERG and oyster contained the highest value. Based on the amount of ERG found in these mushrooms and on a fresh weight basis, a serving of these mushrooms (85 g) would provide between 1-26 mg of ERG (Table 5). Mushrooms, particularly the specialty strains, can serve as an excellent source of ERG.

ORAC_{hydro}, ORACₗᵢₚₒ and ORACₜₒₜₐₗ of the mushrooms are presented in Table 6. The ORAC_{hydro} values of the different mushrooms ranged from 33-131 ,mole TE/g dw, while ORACₗᵢₚₒ values had a narrow range between 5-7 μmole TE/g dw. ORACₜₒₜₐₗ values, calculated by adding the ORAC_{hydro} and ORACₗᵢₚₒ values, ranged between 39-138 μmole TE/g dw.

A. bisporus were found to have the highest values amount the mushrooms tested with a range between 80-131 μmole TE/g dw. The specialty strains provided a range between 33-49 μmole TE/g dw. The ORAC_{hydro} values translate to approximately 5-9 μmole TE/g (fw) depending on the moisture content of the mushrooms. Wu and co-workers analyzed a wide variety of vegetables using the ORAC_{hydro} assay and found a range between 3.0-145 μmole TE/g fw, with beans having the highest values. Many of the vegetables tested had values of less than 15 μmole TE/g fw.

There was no major difference found among the mushrooms in ORACₗᵢₚₒ values. Crimini, maitake and oyster mushrooms had identical values of 5.67 μmole TE/g dw. Both portabella and shiitake mushrooms had the highest ORACₗᵢₚₒ values at 7.00 μmole TE/g dw. The ORACₗᵢₚₒ values translate to approximately 0.48-0.96 μmole TE/g fw depending on the moisture content of the mushrooms. Mushrooms are low in fat with linoleic acid being the most predominate fatty acid; therefore, there may not be a large concentration of lipophilic antioxidant(s) present to produce a high ORACₗᵢₚₒ value.

Wu and coworkers analyzed a wide variety of vegetables using the ORACₗᵢₚₒ assay and found a range between 0.09-4.20 μmole TE/g fw, with beans providing the highest values. Many of the vegetables tested provided values below 1.0 μmole TE/g fw. Generally, ORACₗᵢₚₒ values contribute less than 10% of the ORACₜₒₜₐₗ. However inorder to obtain a complete picture of antioxidant capacity, both hydrophilic and lipophilic components should be tested for ORAC analysis.

When considering the ORACₜₒₜₐₗ valuers there was a significant difference found within the different A. bisporus mushrooms; portabellas was highest and the common white button the lowest value. However, all of the A. bisporus mushrooms were significantly higher than the specialty mushrooms. Among the specialty mushrooms, maitake contained the lowest ORACₜₒₜₐₗ value with shiitake containing the highest. A significant difference was found between shiitake and maitake, however no significant difference was found between oyster and maiitake and also between oyster and shiitake. Genetics (species or strain), growing conditions, and environmental conditions can affect the amount of secondary metabolites produced by the plant. Since the serving size for mushrooms is equivalent to 85 g, (USDA National Nutrient Database for Standard Reference) ORACₜₒₜₐₗ values would range between 564-823 μmole TE/serving.

In addition to testing mushroom samples for ORAC values, ERG was also tested using the ORAC_{hydro} assay and was found to have a value of 6,440 μmole TE/g indicating that it is a powerful scanvenger of ROO radicals. ERG is an odorless, colorless 2-thioimidazole, amino acid that is water soluble up to 0.9 M at room temperature and insoluble in nonpolar solvents. The results of this study concur with other previous studies that have indicated ERG is a scavenger of ROO radicals.

HO. radicals are highly reactive and can be generated via the Fenton reaction. Due to the fact that HO. radicals are short lived with a high rate constant, it is unlikely that antioxidants present at biological concentrations will be able to scavenge the HO radical. However antioxidants, which are able to act as metal chelators, may be able to prevent the formation of the HO. radical, thus acting as a preventative antioxidant. The HORAC assays measures the ability of the antioxidant present to chelate Co(II) prior to the Fenton reaction cocurring. HORAC values of the mushrooms are presented in Table 7. HORAC values ranged between 3.0-13.6 μmole CAE/g dw. There was a significant difference found within the different A. bisporus mushrooms. Portabellas contained the highest value with the common white button containing the lowest value. However, all of the A. bisporus mushrooms were significantly higher than two of the specialty mushrooms (oyster and maitake) with shiitake mushrooms not being significantly different than the white button mushrooms. Among the specialty mushrooms, maitake contained the least HORAC value with shiitake containing the highest. However no significant difference was found among the specialty mushrooms. HORAC values provided a range between 30-81 μmole CAE/85 g serving size. Generally compounds that can chelate metals, such as Co(II) provide high HORAC values.

Previous studies have also indicated that there is no correlation between ORAC values and HORAC values due to the reaction mechanisms of the assay. HORAC values indicate chelating ability to prevent radical formation, while ORAC values indicate peroxyl scavenging ability. Studies have shown that ERG is a powerful scavenger of HO. at high rates. Unliker other scavengers, ERG is able to inhibit iron and copper-ion dependent generation of HO. Through its sulfur atom, ERG has the ability to complex with divalent metal ions; such as copper cadmium and mercury. The HORAC value of ERG was 231 μmole CAE/g. This value is quite high relative to other common natural antioxidants that were previously tested. Overall in vitro data indicates mushrooms, especially portabellas contain antioxidants which are able to chelate metals, such as Co(II) thus preventing HO. radical formation.

Under physiological conditions, the formation of ONOO⁻ can easily result due to the cellular production and interaction of nitric oxide and superoxide radicals. Damage to protein, especially aromatic compounds can result from ONOO⁻. A limited number of papers have been published regarding the antioxidant capacity against ONOO⁻. The NORAC assay is a relatively new assay. SIN-1 is used in the NORAC assay, which when heated decomposes to produce ONOO⁻. DHR-123 (non-fluorescent) is used as the probe, which becomes oxidized by the presence of ONOO⁻ to become rhodamine 123 (fluoresces). If antioxidants present in the sample are able to scavenge ONOO⁻, a delay in fluorescence will occur due to the protection of the probe offered by the antioxidant. Generally, compounds that can scavenge ONOO⁻ will provide high NORAC values. The reaction mechanism for both the ORAC and NORAC assays are very similar since both assays are measuring the capacity of chain-breaking antioxidants.

NORAC values obtained with the mushrooms are presented in Table 8 and they ranged between 2.0-9.0 μmole μmole TE/g dw. There was a significant difference found within the different A. bisporus mushrooms. All of the NORAC values for the A. bisporus mushrooms were significantly higher than the specialty mushrooms and portabellas contained the highest value with the common white button and crimini providing the same values. Among the specialty mushrooms, shiitake had the highest value and was significantly higher than the other specialty mushrooms. NORAC values provided a range between 23-53 μmole TE/85 g serving size. Studies have shown that ERG is a powerful scaventer of ONOO⁻ and is able to provide protection from nitration to biological compounds, such as tyrosine. The NORAC value of ERG was 407 μmole TE/g, which appears to be quite high.

Another biological relevant free radical is O₂⁻ which is less reactive than HO., however O₂⁻ does react very quickly with certain radicals, such as nitric oxide. The SORAC assay is a newly developed assay. Hydroethidine (nonfluorescent) is used as the probe, which is oxidized (fluoresces) in the presence of O₂⁻ that is generated from a xanthine and xanthine oxidase mixture. If antioxidants present in the sample are able to scavenge O₂⁻, a delay in fluorescence will occur due to the protection of the probe offered by the antioxidant. Generally, compounds that can scavenge O₂⁻ will provide high SORAC values. The reaction mechanism for the ORAC, NORAC and SORAC assays are very similar since the assays are measuring the capacity of chain-breaking antioxidants.

SORAC values of the mushrooms are presented in Table 9 and they ranged between 0.37-2.6 kunit SODeq/g dw. Among the A. bisporus mushrooms, portabellas contained the highest value and the common white button contained the lowest value. Among the specialty mushrooms, shiitake had the highest value. Maitake and oyster contained lower values, which were not significantly different from each other. NORAC values provided a range between 3.9-16 kunit SODeq/85 g serving size. Studies have not shown that ERG is a powerful scavenger of O₂⁻. In fact, the NORAC value of ERG was non-detectable. Overall in vitro data indicates mushrooms, primarily portabella and crimini contain antioxidants, which are able to scavenge O₂⁻.

Total phenolics (TP) in all mushroom samples were analyzed using the FCR and the amount in the mushrooms ranged from 4.2-10.6 mg GAE.d dw. (Table 10). The specialty mushrooms all contained amounts ranging from 4.1-4.3 GAE/g dw. All of the A. bisporus mushrooms contained significantly greater TP and ranged between 8.0-10.7 mg GAE/g dw. Portabellas and crimini contained significantly higher TP than white button.

Based on the amount of TP found in these mushrooms and on a fresh weight basis, a serving of these mushrooms (85 g) would provide 43-75 mg of TP (GAE) (Table 7). A higher free radical scavenging activity has been shown in mushrooms that contain higher TP. Cheung and co-workers analyzed shiitake mushrooms and found a positive correlation between total phenolic content in the mushroom extracts (4.79 mg GAE/g dw) and antioxidant capacity. Yang and co-workers analyzed shiitake and oyster mushrooms for TP and found between 6-15 mg/g dw of TP depending on the species of mushroom chosen. The authors concluded that TP were the major occurring antioxidant component found in the mushrooms and contributed significantly to the antioxidant capacity.

The principal soluble phenolic compound found in the skin of mushrooms is γ-L-glutaminyl-4-hydroxybenzene (GHB). The precursors of GHB include chorismic acid, prephenic acid, tyrosine, 4-aminobenzoic acid and 4-amino-phenol. Polymerization of these phenols ultimately results in melanin production, which produces a brown discoloration of mushrooms. Choi and co-workers found A. bisporus to have 5.4 mg/g dw of soluble phenols, as measured by FCR. They analyzed A. bisporus for phenols and found significant amounts of GHB in every part of the fruiting body at a higher concentration than other phenolic acids. Rajarathnam and co-workers investigated the phenolic compounds present in button and oyster mushrooms and found the phenolic content about three times higher in button mushrooms. Based on the ability of phenol oxidase to oxidize various phenolic compounds, the predominate phenolic compounds identified in the button mushrooms included tyrosine, catechol, and the phenolic acids, p-hydroxybenzoic acid, p-coumaric acid and vanillic acid. The oyster mushrooms contained the phenolic compounds, syringaldehyde, guaiacol and catechol with no detection of tyrosine. Maitake mushrooms were analyzed for phenolic acids and were found to be high in p-hydroxy-benzoic acid (66.4 μg/100 g fw) relative to the other phenolic acids identified, tr-cinnamic acid (13.4 μg/100 g fw) and caffeic acid (4.2 μg/100 g fw). The predominate phenolic acids identified in A. bisporus was tr-cinnamic acid (20.7 μg/100 g fw) for the white button and p-hydroxy-benzoic acid (50.3 μg/100 g fw) for crimini. The discoloration of button mushrooms is more extensive as compared to the specialty strains, which is believed to be due to the differences found in the total amount of phenolic compounds and also the diverse functional groups present.

Quantification of TP using FCR is based on electron-transfer reaction, therefore one must consider non-phenolic compounds present in the sample that could contribute to the transfer of electrons, such as vitamin C. A. bisporus, L. edodes, P. ostreatus, and G. frondosa have been analyzed for vitamin C content yielding a range between 17-60 mg/100 g dw, with A. bisporus containing the lowest amount. Due to the fact that A. bisporus was found to have the highest level of TP and along with the fact that this genera of mushrooms do not provide significant amounts of vitamin C, it is unlikely that vitamin C contributes significantly to the TP value, however other non-phenolic compounds may be present that are contributing to the TP value.

The ERG content of the various mushrooms was correlated to the antioxidant potential as measured by the ORACₜₒₜₐₗₛ, HORAC, NORAC and SORAC assays. No significant mathematical correlation was found between ERG and any of the various antioxidant assays (R²=0.47, p>0.05). On the basis of a poor correlation found between the various antioxidant assay values and ERG content, there appears to be no relationship between these parameters in the six cultivated mushrooms measured. However, this does not provide evidence that a poor correlation between ERG content and the antioxidant assays will provide less biological activity.

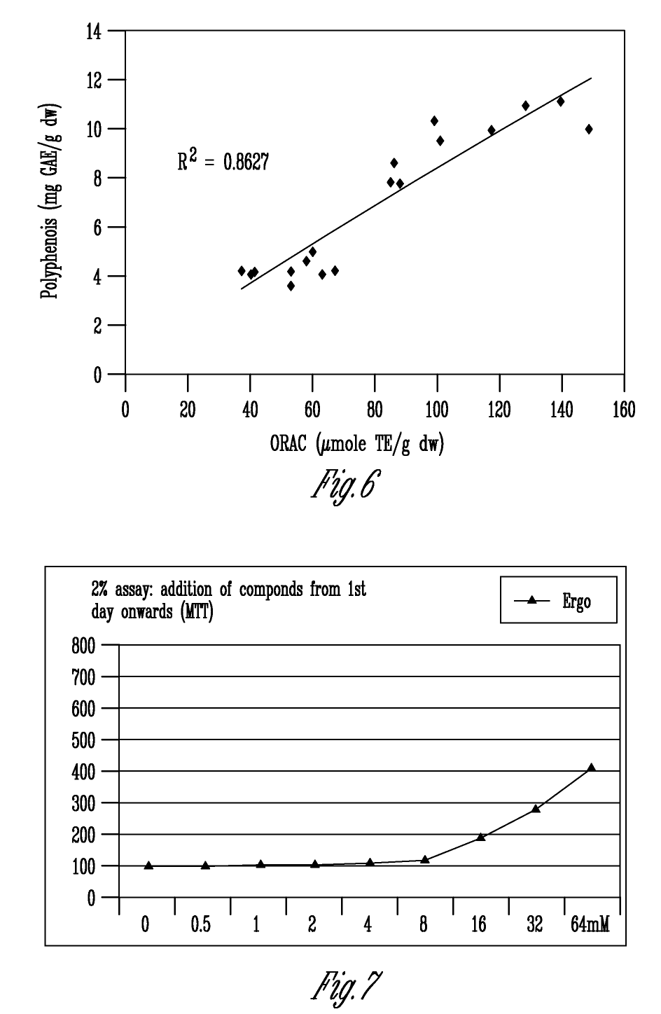

Total phenolics (TP) may account for the antioxidant capacity found among fruits and vegetables. Previous studies have shown a linear correlation between polyphenols and antioxidant capacity (ORAC); however, not all types of foods demonstrate a good correlation. As mentioned earlier, previous studies conducted with mushrooms have shown a correlation between the polyphenol content and antioxidant capacity. Within this study, TP content of the various mushrooms was correlated to the antioxidant potential as measured by the ORACₜₒₜₐₗ, HORAC, NORAC and SORAC assays. No significant mathematical correlation was found between TP and HORAC, NORAC and SORAC assays (p>0.05). However, significant correlation was found between TP and ORACₜₒₜₐₗ (p=0.05). Plotting TP content versus ORACₜₒₜₐₗ (FIG. 6) yields a linear regression with a good correlation (R²=0.863) between these two parameters. Therefore a mathematical relationship exists between TP and ORACₜₒₜₐₗ values indicating that ROO. radicals are scavenged at a greater rate as the TP content of the mushroom increases. One possible mechanism by which phenolic compounds act as antioxidants is through hydrogen donation. The chemistry of the ORAC assay has been shown to proceed through hydrogen atom transfer (HAT) mechanism. Thus, the polyphenols present in the mushrooms are able to donate a hydrogen to the ROO. radicals present.

It has been shown that various genera of edible mushrooms could be viable and economical source of antioxidants in the diet. Also, results of this study indicate that A. bisporus, specifically portabella and crimini, mushrooms have significantly higher antioxidant potential relative to the other mushrooms tested. In addition, TP content in the mushrooms is significantly correlated to ORACₜₒₜₐₗ values. The ramifications of this study could provide valuable new opportunities for mushroom growers, since mushrooms can serve as an excellent source of antioxidants, specifically ERG and TP and provide yet another reason to incorporate mushrooms into the human diet.

TABLE 4
List of Fungi Analyzed for Antioxidant
Capacity (ORAC, HORAC, SORAC, and NORAC assays), Total
Polyphenols (Folin-Colcalteu assay) and Ergothioneine Content.

| Mushrooms species | Sample type | Source |
|---|---|---|
| Agaricus bisporus | White button | Penn State University |
| | (all crops) | |
| | Brown mushroom | Penn State University |
| | (crimini) | |
| | Portabella | Penn State University |
| Lentinula edodes | Basidioma | Modern Mushroom Farm, Inc. |
| Grifola frondosa | Basidioma | Modern Mushroom Farm Inc. |
| | | Penn State University |
| Pleurotus osteratus | Basidioma | Modern Mushroom Farm Inc. |
| Pleurotus eryngii | Basidioma | Modern Mushroom Farm Inc. |

TABLE 5
Concentration of Ergothioneine in Mushrooms

| | mg/g dw of | moisture | mg ERG/ |
|---|---|---|---|
| Sample type | ergothioneine^{a} | (%) | serving^{b} |
| White button | 0.21 ± 0.01^{A} | 92 | 1.4 |
| Crimini | 0.40 ± 0.03^{A} | 91 | 3.1 |
| Portabella | 0.45 ± 0.03^{A} | 93 | 2.7 |
| Maitake | 1.13 ± 0.05^{B} | 83 | 16.3 |
| Shiitake | 1.98 ± 0.11^{C} | 88 | 20.2 |
| Oyster | 2.59 ± 0.18^{D} | 88 | 26.4 |

^{a}mean mg/g dry weight (dw) ± standard deviation for 3 samples tested from one crop followed by different capital letters differ significantly (p = 0.05, Tukey's method).
^{b}Serving size from USDA National Nutrient Database for Standard Reference for Mushrooms (85 g).

TABLE 6
Lipophilic (ORACₗᵢₚₒ), Hydrophilic (ORAC_{hydro}), and ORACₜₒₜₐₗ values for
Mushrooms^{a}

| | moisture | ORACₗᵢₚₒ^{b} | ORAC_{hydro} | ORACₜₒₜₐₗ^{cd} | ORACₜₒₜₐₗ/serving^{e} |
|---|---|---|---|---|---|
| Sample type | (%) | (μmol of TE/g) | (μmol of TE/g) | (μmol of TE/g) | (μmol of TE) |
| White button | 92 | 6.33 ± 0.58 | 80.00 ± 1.7 | 86.33^{A} | 587 |
| Crimini | 91 | 5.67 ± 0.58 | 100.00 ± 9.6 | 105.67^{B} | 808 |
| Portabela | 93 | 7.00 ± 0.00 | 131.33 ± 10.0 | 138.33^{C} | 823 |
| Maitake | 83 | 5.67 ± 0.58 | 33.67 ± 1.5 | 39.33^{D} | 568 |
| Shiitake | 88 | 7.00 ± 0.00 | 55.67 ± 4.5 | 62.67^{E} | 639 |
| Oyster | 88 | 5.67 ± 0.00 | 49.67 ± 3.8 | 55.34^{DE} | 564 |

^{a}Data expressed as dry weight basis and presented as mean ± standard deviation for 3 samples tested from one crop.
^{b}ORAC data expressed as micromoles of Trolox equivalents per gram (μmol of TE/g).
^{c}ORACₜₒₜₐₗ = ORACₗᵢₚₒ + ORAC_{hydro-}
^{d}ORACₜₒₜₐₗ values vollowed by different capital letters differ significantly (p = 0.05, Tukey's method).
^{e}Serving size from USDA National Nutrient Database for Standard Reference for Mushrooms (85 g).

TABLE 7
HORAC values for Mushrooms.

| | HORAC | moisture | μmole CAE/ |
|---|---|---|---|
| Sample type | (μmole CAE/g)^{a} | (%) | serving^{b} |
| White button | 5.33 ± 0.58^{A} | 92 | 36.2 |
| Crimini | 7.67 ± 0.58^{B} | 91 | 58.7 |
| Portabella | 13.67 ± 0.58^{C} | 93 | 81.3 |
| Maitake | 2.67 ± 0.58^{D} | 83 | 38.6 |
| Shiitake | 4.00 ± 0.00^{AD} | 88 | 40.8 |
| Oyster | 3.00 ± 1.00^{D} | 88 | 30.6 |

^{a}Data expressed as dry weight basis and presented as μmole of caffeic acid equivalents per gram (μmole of CAE/g ± standard deviation) for 3 samples tested from one crop followed by different capital letters differ significantly (p = 0.05, Tukey's method).
^{b}Serving size from USDA National Nutrient Database for Standard Reference for Mushrooms (85 g).

TABLE 8
NORAC values for Mushrooms.

| | NORAC | moisture | μmole TE/ |
|---|---|---|---|
| Sample type | (μmole TE/g)^{a} | (%) | serving^{b} |
| White button | 6.33 ± 0.58^{A} | 92 | 43.0 |
| Crimini | 6.33 ± 0.58^{A} | 91 | 43.0 |
| Portabella | 9.00 ± 0.00^{B} | 93 | 53.6 |
| Maitake | 2.00 ± 0.00^{C} | 83 | 28.9 |
| Shiitake | 5.00 ± 0.00^{D} | 88 | 51.0 |
| Oyster | 2.33 ± 0.58^{C} | 88 | 23.8 |

^{a}Data expressed as dry weight basis and presented as μmole of Trolox equivalents per gram (μmole TE/g ± standard deviation) for 3 samples tested from one crop followed by different capital letters differ significantly (p = 0.05, Tukey's method).
^{b}Serving size from USDA National Nutrient Database for Standard Reference for Mushrooms (85 g).

TABLE 9
SORAC values for Mushrooms.

| | SORAC | moisture | kunitSODeq/ |
|---|---|---|---|
| Sample type | (kunitSODeq/g)^{a} | (%) | serving^{b} |
| White button | 0.90 ± 0.10^{A} | 92 | 6.1 |
| Crimini | 1.61 ± 0.11^{B} | 91 | 12.3 |
| Portabella | 2.69 ± 0.11^{C} | 93 | 16.0 |
| Maitake | 0.37 ± 0.04^{D} | 83 | 5.3 |
| Shiitake | 0.77 ± 0.02^{A} | 88 | 7.9 |
| Oyster | 0.38 ± 0.02^{D} | 88 | 3.9 |

^{a}Data expressed as dry weight basis and presented as milligrans of gallic acid equivalents per gram (mg of GAE/g ± standard deviation) for 3 samples tested from one crop followed by different capital letters differ significantly (p = 0.05, Tukey's method).
^{b}Serving size from USDA National Nutrient Database for Standard Reference for Mushrooms (85 g).

TABLE 10
Total Phenolic (TP) values for Mushrooms.

| | TP | moisture | mg GAE/ |
|---|---|---|---|
| Sample type | (mg GAE/g)^{a} | (%) | serving^{b} |
| White button | 8.00 ± 0.48^{A} | 92 | 59.4 |
| Crimini | 9.89 ± 0.43^{B} | 92 | 75.7 |
| Portabella | 10.65 ± 0.61^{B} | 93 | 63.4 |
| Maitake | 4.17 ± 0.06^{C} | 83 | 60.3 |
| Shiitake | 4.32 ± 0.27^{C} | 88 | 44.1 |
| Oyster | 4.27 ± 0.69^{C} | 88 | 43.6 |

^{a}Data expressed as dry weight basis and presented as milligrams of gallic acid equivalents per gram (mg of GAE/g ± standard deviation″) for 3 samples tested from one crop followed by different capital letters differ significantly (p = 0.05, Tukey's method).
^{b}Serving size from USDA National Nutrient Database for Standard Reference for Mushrooms (85 g).

### EXAMPLE 4

Lohman or Hisex Brown chicken one-day-old fertilized eggs were obtained and stored in a lab under appropriate conditions of 12° C. and 80% humidity. At embryonic day 0 eggs were transferred into a breeding incubatore and stored under permanent turning until embryonic day 9 at 37.8° C. and 55% humidity. For isolation of neurons 4 to 6 chicken embryos were used per experiment. Neurons were plated out and nerve cells maintained in a nutrition medium at 37° C., 97% humidity, and 5% CO₂.

Test Item (T.I.) and Reference Item (R.I.) were administered at the start of the experiments for 8 days. Neurons were maintained in the same medium without change or addition of media until the end of the experiments. During the whole culture period of 8 days cells stay in an incubator at 37° C., 97% humidity, and 5% CO₂.

From T.I.1, L-Ergothioneine a 128 mM solution was prepared in medium and directly added to the cells at the start of the experiments for a total of 8 days. In case of R.I.1, Mangostin, 50 mg of the powder were dissolved in 10 ml aqua bidest; this 5mg/ml stock-solution was centrifuged for 15 min with 3000 rpm. The pellet was discarded and only from the supernatant 1.25 μl up to 160 μl/ml medium was administered. R.I.2. the neurotrophic standard NTS is a ready to use injection solution and was applied in concentrations from 1.25 μl up ro 160 μl/ml medium. The stock-solution for R.I.3, Trolox, was made in a concentration of 2 mM in PBS and the appropriate concentrations added to the cells at day 1.

To the isolated nerve cell T.I.1 was added in a dose range of 0.5, 1, 2, 4, 8, 16, 32, 64 mM. The supernatant of R.I.1 and the ready to use injection solution of R.I.2 were applied in the concentration 1.25, 2.5, 5, 10, 20, 40, 80, 160 μl/ml medium. R.I.3 was tested in the concentrations of 1, 2, 4, 8, 16, 31.25, 62.5, 125 μM. Experiments were performed at two different days, whereby on each day two identical microplates were prepared with two to four wells per T.I. or R.I. (n=8). In this 2% cell stress assay substances were administered at start of the experiment (day 1 in vitro) for the whole experimental period that means for eight days in vitro.

Neuronal viability of cultures was determined with the MTT-assay using a plate-reader (570 nm) as described in SOP MET009. The MTT-assay is a sensitive assay measuring the mitochondrial dehydrogenase activity in viable cells only. It is performed according to the method described by Mosmann, J. Immunol. Meth., 1983, 55-63. This assay is based on the reduction of yellow MTT (3-(4,5-dimethylthiazol-2-yl)-2,5,diphenyl tetrazolium bromide), to dark blue formazan crystals by mitochondrial dehydrogenases (succinate dehydrogenase). Since this reaction is catalyzed in living cells only the assay can be used for the quantification of cell viability.

For the determination of cell viability MTT solution was added to each well in a final concentration of 0.5 mg/ml. After 2 hours the MTT containing medium was aspired. Cells were lysed with 3% SDS, formazan crystals dissolved in isopropanol/HCI. To estimate optical density a plate reader (Anthos HT II) was used at wavelength 570 nm. Neuronal viability is expressed as optical densities (ODs).

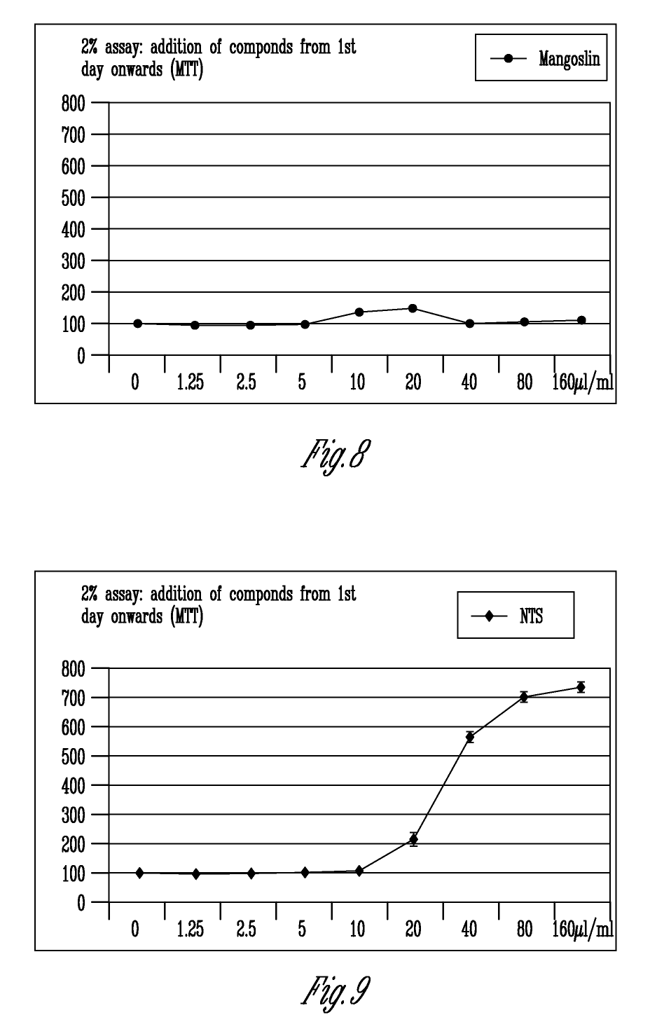

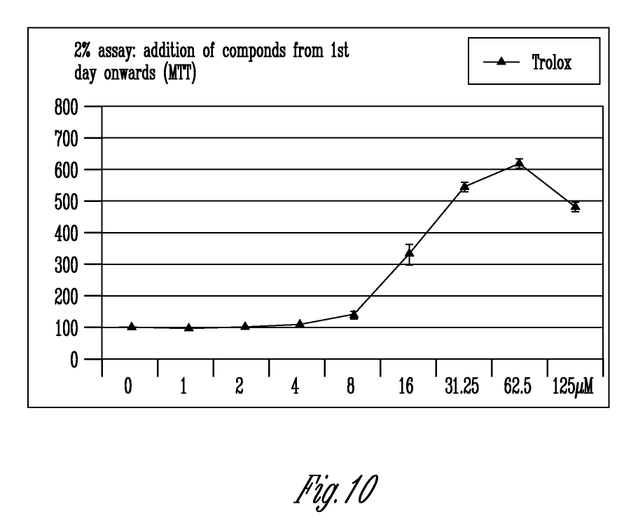

The results obtained with the T.I. Ergothioneine and the R.I. Mangostin, NTS and Trolox are shown in Tables 11 and 12 as well as in FIGS. 7 to 10. Table 13 shows the p-values obtained with the students T-test when evaluating the differences between the compounds in different concentrations versus the untreated control. Table 11 displays the optical density measurement results as mean standard deviation and sem of two independent experiments performed on two days with a total of n=8 for each compound concentration and a n=64 for the untreated control. Table 12 shows the same results calculated as percentage of the untreated control for which after 8 days of serum=growth factor withdrawal viability of the remaining neurons has been assumed to be 100%. FIGS. 7 to 10 show the separate results of each of the test compounds investigated, starting with Ergo (FIG. 7). From 4 mM onwards this radical scavenger exhibits its neuroprotective potential. Compared to Mangostin (FIG. 8) which is neuroprotective from 10 to 160 μl/ml medium (+155%) effect magnitude of Ergo with 407% viability is higher. However it is much lower compared to NTS (FIG. 9) which is reaching 732% of neuronal viability, showing a classical dose response profile and significant neuroprotective properties between 10 and 160 μl/ml medium. The effects of the soluble vitamin E, Trolax, are shown in FIG. 10. Other than Ergo, which is active in the high millimolar range, Trolox is active in the micro molar range between 4 μM and 125 μM, reaching a maximum of 620% neuronal vitality with 62.5 μM. Effects obtained with Trolox also indicate that the 2% Low Serum assay is a good tool to test neuroprotective effect which might be due to the radical scavenging properties of a compound. The divergences in the dose range profile between Ergo and Trolox, both described to exhibit radical scavenging properties might be the result of an activity loss of Ergo due to the well known stability problems of radical scavengers. This is even more likely since the batch used was stored under normal conditions for a longer period before testing in this assay. It is thought that a “fresh” batch might not even increase the effect magnitude but also shift the dose response profile into the micro molar range.

The data shows that treatment of chicken embryo telencephalon neurons with ergothioneine demonstrated a significant increase in the viability of cortical neurons as measured by mitchondiral dehydrogenase activity over control cells. Specifically, the results obtained with the radical scavenger Ergothioneine, and with the references of Mangostin, the neurotrophic standard NTS, a peptide mixture with clear neuroprotective potential, and with the water soluble vitamin E, Trolox described to have radical scavenging properties, indicate a clear neurotrophic and/or neuroprotective effect for all compounds/preparations measured. The peptide mixture NTS was most effective reaching a maximum viability effect of 702% followed by Trolox with 620%, by Erothioneine with 407% and Mangostin with 155% and therefore lower compared to all other compounds. Compared to Trolox which was protective in micromolar concentrations, Ergothioneine which is active in the high millimolar range seems to have lost at least part of its activity dut to stability problems.

TABLE 11
2% Low Serum Assay: Effects of T.I. and R.I. on viability of cortical neurons
obtained with the MTT method. T.I. and R.I. addition at 1DIV. Results shown as mean
and standard deviation and SEM of the optical densities (OD):
mean

| | | | | | | | | | |
|---|---|---|---|---|---|---|---|---|---|
| Ergo | 0 | 0.5 | 1 | 2 | 4 | 8 | 16 | 32 | 64 mM |
| | 0.054 | 0.053 | 0.057 | 0.057 | 0.060 | 0.065 | 0.103 | 0.153 | 0.221 |
| Mangostin | 0 | 1.25 | 2.5 | 5 | 10 | 20 | 40 | 80 | 160 μl/ml |
| | 0.054 | 0.051 | 0.052 | 0.053 | 0.076 | 0.064 | 0.057 | 0.059 | 0.065 |
| NTS | 0 | 1.25 | 2.5 | 5 | 10 | 120 | 40 | 80 | 160 μl/ml |
| | 0.057 | 0.063 | 0.057 | 0.056 | 0.062 | 0.125 | 0.321 | 0.401 | 0.418 |
| Trolox | 0 | 1 | 2 | 4 | 8 | 16 | 31.25 | 62.5 | 125 μM |
| | 0.054 | 0.052 | 0.056 | 0.058 | 0.076 | 0.180 | 0.297 | 0.338 | 0.262 |

stand. dev.

| | | | | | | | | | |
|---|---|---|---|---|---|---|---|---|---|
| Ergo | 0 | 0.5 | 1 | 2 | 4 | 8 | 16 | 32 | 64 mM |
| | 0.003 | 0.003 | 0.002 | 0.003 | 0.003 | 0.003 | 0.020 | 0.027 | 0.015 |
| Mangostin | 0 | 1.25 | 2.5 | 5 | 10 | 20 | 40 | 80 | 160 μl/ml |
| | 0.003 | 0.003 | 0.002 | 0.003 | 0.025 | 0.016 | 0.004 | 0.002 | 0.002 |
| NTS | 0 | 1.25 | 2.5 | 5 | 10 | 120 | 40 | 80 | 160 μl/ml |
| | 0.004 | 0.003 | 0.004 | 0.003 | 0.004 | 0.039 | 0.013 | 0.014 | 0.011 |
| Trolox | 0 | 1 | 2 | 4 | 8 | 16 | 31.25 | 62.5 | 125 μM |
| | 0.004 | 0.002 | 0.004 | 0.004 | 0.015 | 0.050 | 0.021 | 0.026 | 0.025 |

sem

| | | | | | | | | | |
|---|---|---|---|---|---|---|---|---|---|
| Ergo | 0 | 0.5 | 1 | 2 | 4 | 8 | 16 | 32 | 64 mM |
| | 0.001 | 0.001 | 0.001 | 0.001 | 0.001 | 0.001 | 0.007 | 0.010 | 0.005 |
| Mangostin | 0 | 1.25 | 2.5 | 5 | 10 | 20 | 40 | 80 | 160 μl/ml |
| | 0.001 | 0.001 | 0.001 | 0.001 | 0.009 | 0.006 | 0.001 | 0.001 | 0.001 |
| NTS | 0 | 1.25 | 2.5 | 5 | 10 | 120 | 40 | 80 | 160 μl/ml |
| | 0.001 | 0.001 | 0.002 | 0.001 | 0.001 | 0.014 | 0.005 | 0.005 | 0.004 |
| Trolox | 0 | 1 | 2 | 4 | 8 | 16 | 31.25 | 62.5 | 125 μM |
| | 0.001 | 0.001 | 0.001 | 0.001 | 0.005 | 0.018 | 0.008 | 0.009 | 0.009 |

Values represent the mean, standard deviations and SEM of ODs from two independent experiments (12790A and 12790B) performed at two days with two 96-well plates (n=8 for each T.I. and R.I. concentration and n=64 for controls). The control is represented by 0 and did not receive any T.I. or R.I.

TABLE 12
2% Low Serum Assay: Effects of T.I. and R.I. on viability of cortical neurons
obtained with the MTT method. T.I. and R.I. addition at 1DIV. Results shown as mean
and standard deviation and SEM of the data in % (control [0 μM] is 100%):
mean

| | | | | | | | | | |
|---|---|---|---|---|---|---|---|---|---|
| Ergo | 0 | 0.5 | 1 | 2 | 4 | 8 | 16 | 32 | 64 mM |
| | 100.00 | 98.13 | 104.42 | 105.10 | 110.39 | 119.84 | 189.48 | 262.66 | 407.45 |
| Mangostin | 0 | 1.25 | 2.5 | 5 | 10 | 20 | 40 | 80 | 160 μl/ml |
| | 100.00 | 93.86 | 95.97 | 98.32 | 142.18 | 155.61 | 105.20 | 109.66 | 120.33 |
| NTS | 0 | 1.25 | 2.5 | 5 | 10 | 120 | 40 | 80 | 160 μl/ml |
| | 100.00 | 91.95 | 99.48 | 97.81 | 107.94 | 217.40 | 562.85 | 702.09 | 731.78 |
| Trolox | 0 | 1 | 2 | 4 | 8 | 16 | 31.25 | 62.5 | 125 μM |
| | 100.00 | 97.79 | 102.23 | 107.05 | 140.44 | 330.68 | 546.15 | 620.23 | 481.52 |

stand. dev.

| | | | | | | | | | |
|---|---|---|---|---|---|---|---|---|---|
| Ergo | 0 | 0.5 | 1 | 2 | 4 | 8 | 16 | 32 | 64 mM |
| | 5.29 | 4.30 | 2.61 | 4.42 | 5.07 | 3.81 | 36.45 | 50.50 | 15.88 |
| Mangostin | 0 | 1.25 | 2.5 | 5 | 10 | 20 | 40 | 80 | 160 μl/ml |
| | 4.62 | 4.91 | 3.47 | 5.67 | 49.36 | 28.18 | 6.27 | 3.36 | 2.86 |
| NTS | 0 | 1.25 | 2.5 | 5 | 10 | 120 | 40 | 80 | 160 μl/ml |
| | 5.68 | 7.00 | 10.00 | 6.44 | 8.84 | 64.59 | 41.89 | 31.97 | 31.49 |
| Trolox | 0 | 1 | 2 | 4 | 8 | 16 | 31.25 | 62.5 | 125 μM |
| | 6.09 | 4.25 | 7.11 | 8.07 | 29.25 | 92.83 | 35.44 | 50.11 | 42.35 |

sem

| | | | | | | | | | |
|---|---|---|---|---|---|---|---|---|---|
| Ergo | 0 | 0.5 | 1 | 2 | 4 | 8 | 16 | 32 | 64 mM |
| | 1.32 | 1.52 | 0.92 | 1.56 | 1.79 | 1.35 | 12.89 | 17.85 | 5.61 |
| Mangostin | 0 | 1.25 | 2.5 | 5 | 10 | 20 | 40 | 80 | 160 μl/ml |
| | 1.16 | 1.74 | 1.23 | 2.00 | 17.45 | 9.86 | 2.22 | 1.19 | 1.01 |
| NTS | 0 | 1.25 | 2.5 | 5 | 10 | 120 | 40 | 80 | 160 μl/ml |
| | 1.42 | 2.48 | 3.54 | 2.28 | 3.13 | 22.84 | 14.81 | 11.30 | 11.13 |
| Trolox | 0 | 1 | 2 | 4 | 8 | 16 | 31.25 | 62.5 | 125 μM |
| | 1.52 | 1.50 | 2.51 | 2.85 | 10.34 | 32.82 | 12.53 | 17.72 | 14.97 |

Values represent the mean, standard deviations and SEM in % (100% =untreated control) from two independent experiments (12790A and 12790B) performed at two days with two 96-well plates (n=8 for each T.I. and R.I. concentration and n=64 for controls). The control is represented by 0 and did not receive any T.I. or R.I.

TABLE 13
2% Low Serum Assay: Students T-test results (p-values) showing statistical
differences (red, p ≦ 0.05) between the T.I. Ergo and the R.I. Mangostin, NTS and Trolox
versus the untreated control (0).
differences versus control

| | | | | | | | | |
|---|---|---|---|---|---|---|---|---|
| Ergo | 0.5 | 1 | 2 | 4 | 8 | 16 | 32 | 64 mM |
| | 0.4904063 | 0.0731448 | 0.0565226 | 0.0006532 | 6.667E−08 | 2.051E−09 | 8.861E−13 | 9.029E−23 |
| Mangostin | 1.25 | 2.5 | 5 | 10 | 20 | 40 | 80 | 160 μl/ml |
| | 0.0092997 | 0.0450405 | 0.4209283 | 0.0015281 | 1.876E−07 | 0.0483116 | 6.395E−05 | 7.868E−10 |
| NTS | 1.25 | 2.5 | 5 | 10 | 120 | 40 | 80 | 160 μl/ml |
| | 0.0075899 | 0.8039988 | 0.4245646 | 0.0166953 | 4.324E−07 | 6.431E−28 | 6.815E−30 | 1.329E−32 |
| Trolox | 1 | 2 | 4 | 8 | 16 | 31.25 | 62.5 | 125 μM |
| | 0.1000095 | 0.4452303 | 0.0273856 | 1.43E−05 | 9.126E−10 | 3.183E−23 | 5.902E−23 | 2.922E−20 |

What is claimed is:

1. A novel organic form of ergothioneine and/or selenium having the following formula:

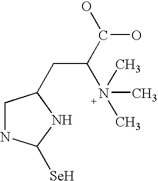

2. The ergothioneine/selenium of claim 1 wherein said ergothioneine/selenium is purified from mushrooms.

3. A pharmaceutical composition for treating a disease state or condition associated with neurodegeneration such as stroke, head trauma, subarachnoid hemorrhage, radiation damage, Alzheimer's or Parkinson's disease comprising:
a therapeutically effective amount of L-ergothioneine and a carrier.

4. A pharmaceutical composition for treating a disease state or condition associated with neurodegeneration such as stroke, head trauma, subarachnoid hemorrhage, radiation damage, Alzheimer's or Parkinson's disease, comprising:
administering to an animal a therapeutically effective amount of L-ergothioneine and a carrier.

5. A pharmaceutical composition for prophylactic treatment of a disease state or condition associated with neurodegeneration such as stroke, head trauma, subarachnoid hemorrhage, radiation damage, Alzheimer's or Parkinson's disease, comprising:
a therapeutically effective amount of L-ergothioneine and a carrier.

6. A pharmaceutical composition for prophylactic treatment of a disease state or condition associated with neurodegeneration such as stroke, head trauma, subarachnoid hemorrhage, radiation damage, Alzheimer's or Parkinson's disease, comprising:
administering to an animal a therapeutically effective amount of L-ergothioneine and a carrier.
